(19) United States
(12) Patent Application Publication
(10) Pub. No.: US 2020/0048347 A1
(43) Pub. Date: Feb. 13, 2020

(54) BISPECIFIC ANTIBODIES

(71) Applicant: ImmunoMab, Inc., Richmond, CA (US)

(72) Inventors: Zhenwei MIAO, Richmond, CA (US); Fen ZHANG, Richmond, CA (US); Jennifer LU, Richmond, CA (US)

(21) Appl. No.: 16/329,701

(22) PCT Filed: Aug. 30, 2017
(86) PCT No.: PCT/US2017/049472

§ 371 Date: Feb. 28, 2019

Related U.S. Application Data
(60) Provisional application No. 62/382,495, filed on Sep. 1, 2016.

(51) Int. Cl. C07K 16/28; C07K 16/22; C07K 16/24; C07K 16/30; C07K 16/46

(52) U.S. Cl. CPC C07K 16/2809; C07K 16/22; C07K 16/244; C07K 16/2887; C07K 2317/73; C07K 16/468; C07K 2317/24; C07K 2317/31; C07K 16/303

(57) ABSTRACT

In certain aspects, bispecific antibodies and uses thereof are provided.

### CROSS REFERENCE TO RELATED APPLICATIONS

This application claims the benefit of U.S. Provisional Application 62/382,495, filed on Sep. 1, 2016, which is incorporated herein by reference in its entirety.

### REFERENCE TO SEQUENCE LISTING

The present application is being filed along with a Sequence Listing in electronic format. The Sequence Listing is provided as a file entitled IMMB_001WO.TXT, created Aug. 30, 2017, which is 87 kb in size. The information in the electronic format of the Sequence Listing is incorporated herein by reference in its entirety.

### BACKGROUND

Bispecific antibodies can provide therapeutic benefits in the treatment of various conditions, including various cancers by targeting two different moieties. Accordingly, it is desirable to provide bispecific antibodies with therapeutically useful properties.

### SUMMARY

Some embodiments provide a bispecific antibody including at least one synthetic linker, methods of preparing the bispecific antibody including at least one synthetic linker, and uses thereof, wherein a heavy chain antibody portion includes the amino acid sequence SPPC, CPPS, APPC or CPPA in the hinge region wherein the cysteine sulfur of the SPPC, CPPS, APPC or CPPA sequence is covalently bonded to at least one synthetic linker.

Some embodiments provide a bispecific antibody including at least one synthetic linker, methods of preparing the bispecific antibody including at least one synthetic linker, and uses thereof, wherein the at least one synthetic linker includes

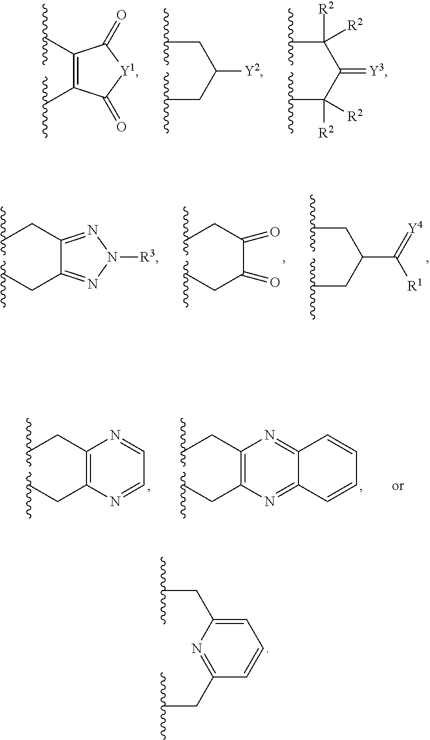

### BRIEF DESCRIPTION OF THE DRAWINGS

FIG. 1 provides a bispecific antibody synthesized using six different bifunctional linker reagents.

FIG. 2 provides an HIC-HPLC trace of the bispecific antibody of Table 12.

FIG. 3 provides the HIC-HPLC trace of a mixture of the bispecific antibody of Table 12 with Adalimumab antibody and Anti-IL17 antibody.

FIG. 4 provides the HIC-HPLC trace of a mixture of purified bispecific antibody of Table 12 with Adalimumab antibody and Anti-IL17 antibody.

FIG. 5 provides Bio-Analyzer Analysis comparing bispecific antibody of Table 12 in a mixture with Adalimumab antibody and Anti-IL17 antibody.

FIG. 6 provides an HPLC trace of the bispecific antibody of Table 13 in a mixture with Bevacizumab antibody and Anti-IL17 antibody.

FIG. 7 provides a SDS gel of the bispecific antibody of Table 13 in reduced and non-reduced form.

FIG. 8 provides the RPLC trace of a mixture of the bispecific antibody of Table 14 with Bevacizumab and Anti-Ang2.

FIG. 9 provides the SEC-HPLC of a mixture of the bispecific antibody of Table 14 with Bevacizumab antibody and Anti-Ang2 antibody.

FIG. 10 provides SDS Page comparing bispecific antibody of Table 14 with Bevacizumab antibody and Anti-Ang2 antibody.

FIG. 11A provides a graph demonstrating the bispecific antibody of Table 14 binds to Binds to two Antigens.

FIG. 11B provides a graph demonstrating the bispecific antibody of Table 14 binds to Binds to two Antigens.

FIG. 12 provides the RPLC trace of the bispecific antibody of Table 15.

FIG. 13 provides Bio-Analyzer Analysis comparing bispecific antibody of Table 15 with Anti-HGF and Bevacizumab, each in reduced and non-reduced form.

FIG. 14 provides the HIC-HPLC trace of the bispecific antibody of Table 16.

FIG. 15 provides the HIC-HPLC of a mixture of the bispecific antibody of Table 16 with a mixture of Anti-CD3 antibody and Anti-CD20 antibody.

FIG. 16 provides the HIC-HPLC trace of the bispecific antibody of Table 17 with a mixture of Anti-CD3 antibody and Anti-5T4 antibody.

FIG. 17 provides the SEC HPLC trace of the bispecific antibody of Table 17.

FIG. 18 provides the HIC-HPLC trace of the bispecific antibody of Table 18.

FIG. 19 provides the HIC-HPLC trace of the bispecific antibody of Table 19 with Anti-CD47 antibody and Anti-5T4 antibody.

FIG. 20 provides the SEC HPLC trace of the bispecific antibody of Table 19.

FIG. 21 provides the HIC-FPLC trace of a large scale synthesis of the bispecific antibody of Table 19 with a mixture of Anti-CD47 antibody and Anti-5T4 antibody.

FIG. 22 provides the HIC-FPLC trace of a large scale synthesis of the bispecific antibody of Table 19.

FIG. 23 provides the SEC-FPLC trace of a large scale synthesis of the bispecific antibody of Table 19.

FIG. 24 provides the SDS page of a large scale synthesis of the bispecific antibody of Table 19 in reduced and non-reduced form.

FIG. 25 provides the RP-HPLC trace of the bispecific antibody of Table 20 with a mixture of Anti-CD47 antibody and Anti-GPC3 antibody.

FIG. 26 provides the SEC HPLC trace of the bispecific antibody of Table 20 with a mixture of Anti-CD47 antibody and Anti-GPC3 antibody.

FIG. 27 provides the HIC-HPLC of the bispecific antibody of Table 21 with a mixture of Anti-CD47 antibody and Anti-mesothelin antibody.

FIG. 28 provides a SDS page gel.

FIG. 29A provides a graph of Cytotoxicity Assay Results of Bispecific CD3-Mesothelin.

FIG. 29B a graph of Cytotoxicity Assay Results of Bispecific CD3-Mesothelin.

FIG. 30 shows a graph of the effect of effector to target cell ratios on cytotoxicities of bispecific antibody against triple negative cancer cells.

FIG. 31A shows s graph of the cytotoxicities of bispecific antibodies against pancreatic cancer cells.

FIG. 31B shows a graph of the cytotoxicities of bispecific antibodies against pancreatic cancer cells.

FIG. 32A-D shows graphs of the effect of 5T4-CD47 bispecific antibody versus 5T4 and CD47 antibody is measured in MDA468 (triple negative breast cancer) cells and PBMCs. MFI was measured for 5T4-CD47, 5T4, CD47, and HuIgG antibodies across about −2 to 3 log antibody (nM) for MDA468 cells.

FIG. 33A-D shows graphs of the effect of 5T4-CD47 bispecific antibody versus 5T4 and CD47 antibody is measured in PA-1 (ovarian cancer) cells and PBMCs.

FIG. 34A-D shows graphs of the effect of 5T4-CD47 and 5T4-CD3 bispecific antibodies versus 5T4 and CD47 antibody is measured in DU-145 (prostate cancer) cells and PBMCs.

FIG. 35A-B shows graphs of the effect of treating MDA 231 triple negative breast cancer cells with 1 nM of various antibodies.

FIG. 36A-B shows graphs of the effect of treating Lovo (colon cancer) cells with 1 nM of various antibodies.

FIG. 37A-C shows graphs and pictures demonstrating the effect of treatment with CD47-5T4 antibodies on tumor growth.

FIG. 38 shows a graph of the effect of treating MDA 231 (triple negative breast cancer) cells with various antibodies.

### DETAILED DESCRIPTION

Some embodiments provide a bispecific antibody, comprising a light chain (LC1) and heavy chain (HC1) of an antibody (Ab1) targeting a first moiety; and a light chain (LC2) and heavy chain (HC2) of an antibody (Ab2) targeting a second moiety, wherein LC1 and HC1 may be connected to each other via a linker to provide a first subunit, wherein LC2 and HC2 may be connected to each other via a linker to provide a second subunit, and wherein the first subunit and the second subunit may be connected to each other via a linker.

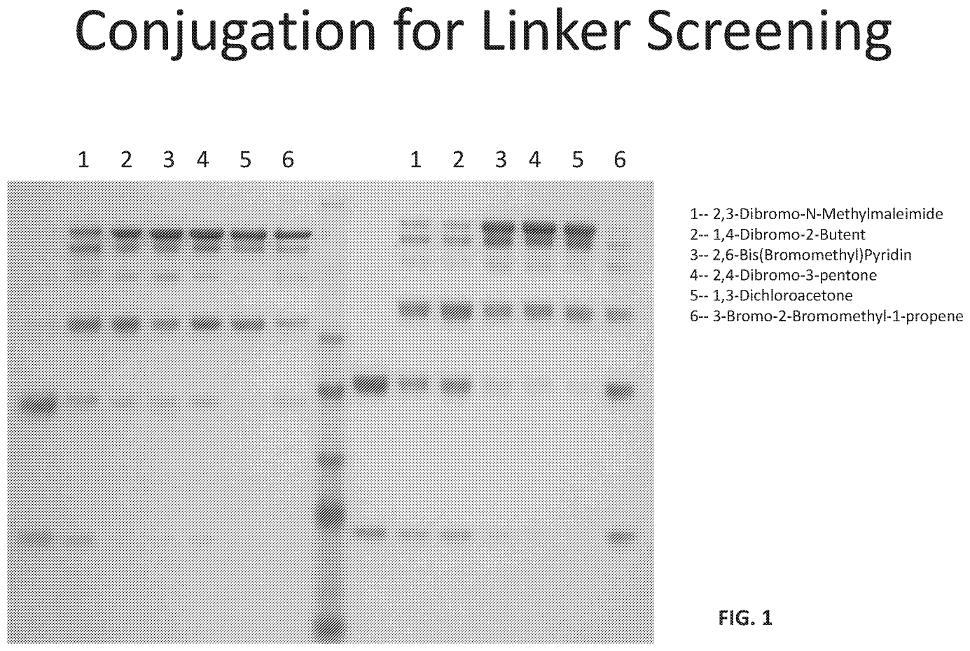

The term “antibody” as used herein refers to whole, monoclonal antibodies. Such whole antibodies consist of two pairs of a “light chain” (LC) and a “heavy chain” (HC) (such light chain (LC)/heavy chain pairs are abbreviated herein as LC/HC). The light chains and heavy chains of such antibodies are polypeptides consisting of several domains. In a whole antibody, each heavy chain comprises a heavy chain variable region (abbreviated herein as HCVR or VH) and a heavy chain constant region. The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain VL and a light chain constant domain CL. The structure of one naturally occurring whole antibody, the IgG antibody, is shown e.g. in FIG. 1. The variable domains VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The two pairs of heavy chain and light chain (HC/LC) are capable of specifically binding to same antigen. Thus said whole antibody is a bivalent, monospecific antibody. Such “antibodies” include e.g. mouse antibodies, human antibodies, chimeric antibodies, humanized antibodies and genetically engineered antibodies (variant or mutant antibodies) as long as their characteristic properties are retained. Especially preferred are human or humanized antibodies, especially as recombinant human or humanized antibodies. In some embodiments, the antibody may be Adalimumab, Bevacizumab, Anti-IL17, Anti-PDGF, Anti-Ang2, Anti-HGF, Anti-CD3, Anti-CD20, anti-CLL1, Anti-mesothelin. Anti-CD47, Anti-5T4, Anti-Trop2 or Anti-GPC3.

There are five types of mammalian antibody heavy chains denoted by the Greek letters: α, δ, ε, γ, and μ. The type of heavy chain present defines the class of antibody; these chains are found in IgA, IgD, IgE, IgG, and IgM antibodies, respectively. Distinct heavy chains differ in size and composition; α and γ contain approximately 450 amino acids, while μ and ε have approximately 550 amino acids.

Each heavy chain has two regions, the constant region and the variable region. The constant region is identical in all antibodies of the same isotype, but differs in antibodies of different isotype. Heavy chains γ, α and δ have a constant region composed of three constant domains CH1, CH2, and CH3 (in a line), and a hinge region for added flexibility; heavy chains and μ have ε constant region composed of four constant domains CH1, CH2, CH3, and CH4. The variable region of the heavy chain differs in antibodies produced by different B cells, but is the same for all antibodies produced by a single B cell or B cell clone. The variable region of each heavy chain is approximately 110 amino acids long and is composed of a single antibody domain.

In mammals there are only two types of light chain, which are called lambda (λ) and kappa (κ). A light chain has two successive domains: one constant domain CL and one variable domain VL. The approximate length of a light chain is 211 to 217 amino acids. Preferably the light chain is a kappa (κ) light chain, and the constant domain CL is preferably C kappa (κ).

The terms “monoclonal antibody” or “monoclonal antibody composition” as used herein refers to a preparation of antibody molecules of a single amino acid composition.

In some embodiments, the “antibodies” may be of any class (e.g. IgA, IgD, IgE, IgG, and IgM, preferably IgG or IgE), or subclass (e.g., IgG1, IgG2, IgG3, IgG4, IgA1 and IgA2, preferably IgG1).

The term “chimeric antibody” as used herein refers to an antibody comprising a variable region, i.e., binding region, from one source or species and at least a portion of a constant region derived from a different source or species, usually prepared by recombinant DNA techniques. In some embodiments, the chimeric antibodies may comprise a murine variable region and a human constant region. In some embodiments, the chimeric antibodies are those in which the constant region has been modified or changed from that of the original antibody, especially in regard to C1q binding and/or Fc receptor (FcR) binding. Such chimeric antibodies are also referred to as “class-switched antibodies.” In some embodiments, the chimeric antibodies are the product of expressed immunoglobulin genes comprising DNA segments encoding immunoglobulin variable regions and DNA segments encoding immunoglobulin constant regions.

The term “humanized antibody” refers to antibodies in which the framework or “complementarity determining regions” (CDR) have been modified to comprise the CDR of an immunoglobulin of different specificity as compared to that of the parent immunoglobulin. In some embodiments, a murine CDR may be grafted into the framework region of a human antibody to prepare the “humanized antibody.” In some embodiments, the “humanized antibodies” are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge. Human antibodies can also be produced in phage display libraries.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable region of a heavy chain (VH)) as used herein denotes each of the pair of light and heavy chains which is involved directly in binding the antibody to the antigen.

The terms “hypervariable region” or “antigen-binding portion of an antibody” as used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids.

The “constant domains” of the heavy chain and of the light chain are not involved directly in binding of an antibody to an antigen, but exhibit various effector functions. Depending on the amino acid sequence of the constant region of their heavy chains, antibodies or immunoglobulins are divided into the classes:

The term “bivalent, bispecific antibody” as used herein refers to an antibody as described above in which each of the two pairs of heavy chain and light chain (HC/LC) binds specifically to a different antigen, i.e. the first heavy and the first light chain (originating from an antibody against a first antigen) together bind specifically to a first antigen, and, the second heavy and the second light chain (originating from an antibody against a second antigen) together bind specifically to a second antigen; such bivalent, bispecific antibodies are capable of specifically binding to two different antigens at the same time, and typically not to more than two antigens. This is in contrast to, on the one hand a monospecific antibody capable of binding only to one antigen, and on the other hand e.g. a tetravalent, tetraspecific antibody which can bind to four antigen molecules at the same time.

Some embodiments provide a bispecific antibody represented by the following structural formula (I):

wherein:
LCA may be a light chain antibody portion;
HCA may be a heavy chain antibody portion;
LCB may be a light chain antibody portion;
HCB may be a heavy chain antibody portion;
L¹ may be a synthetic linker, L² may be a synthetic linker, and L³ may be a synthetic linker; and HCA and HCB each may include the amino acid sequence SPPC, CPPS, APPC or CPPA in the hinge region wherein the cysteine sulfur of the SPPC, CPPS, APPC or CPPA sequence of HCA and HCB is covalently bonded to L³, wherein LCA and LCB are not derived from the same antibody. Example amino acid sequences of such HCs modified in the hinge region to contain the sequence SPPC, CPPS, APPC or CPPA are shown in the sequences provided herewith.

Some embodiments provide a bispecific antibodies represented by the following structural formula (II):

wherein:

LCA may be a light chain antibody portion;

HCA may be a heavy chain antibody portion;

LCB may be a light chain antibody portion;

HCB may be a heavy chain antibody portion;

L¹, L² and L³ may include

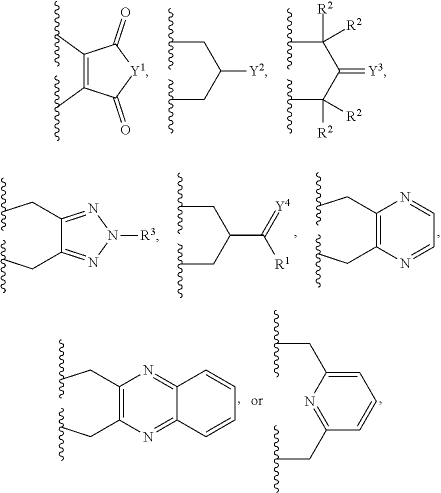

wherein:

Y¹ may be O (oxygen), NR⁴, —NH—NH—, or —CH═CH—;

Y² may be OH or C₁₋₆ alkoxy;

Y³ may be O (oxygen), N—OR⁴, or —CF₂—;

Y⁴ may be O (oxygen), N—OR⁴, or —CF₂—;

R¹ may be H (hydrogen), C₁₋₆ alkyl, aryl, or heteroaryl;

R² may be H (hydrogen) or C₁₋₆ alkyl;

R³ may be H (hydrogen) or C₁₋₆ alkyl; and

R⁴ may be H (hydrogen) or C₁₋₆ alkyl,

wherein LCA and LCB are not derived from the same antibody.

In some embodiments of the bispecific antibodies represented by structural formula (II), HCA and HCB may each include the amino acid sequence SPPC, CPPS, APPC or CPPA in the hinge region wherein the cysteine sulfur of the SPPC, CPPS, APPC or CPPA sequence of HCA and HCB may be covalently bonded to L³.

In some embodiments of the bispecific antibodies represented by structural formula (II), HCA and HCB may each include the amino acid sequence SPPC, CPPS, APPC or CPPA in the hinge region wherein the cysteine sulfur of the SPPC, CPPS, APPC or CPPA sequence of HCA and HCB may be covalently bonded to L³, wherein LCA and LCB are not derived from the same antibody.

In some embodiments of the bispecific antibodies represented by structural formula (II) at least one amino acid residue in the hinge region of HCA or HCB may be replaced with at least one cysteine residue.

In some embodiments of the bispecific antibodies represented by structural formula (II) at least one amino acid residue in the hinge region of HCA and HCB may be replaced with at least one cysteine residue.

In some embodiments of the bispecific antibodies represented by structural formula (II) at least two amino acid residues in the hinge region of HCA and HCB may each be replaced with a cysteine residue.

In some embodiments of the bispecific antibodies represented by structural formula (II) at least one amino acid residue in the hinge region of HCA and HCB may be replaced with at least one cysteine residue, wherein the hinge region of HCA and/or HCB comprises amino acids 210-250 (EU numbering system).

In some embodiments of the bispecific antibodies represented by structural formula (II) at least two amino acid residues in the hinge region of HCA and HCB may each be replaced with a cysteine residue, wherein the hinge region of HCA and/or HCB comprises amino acids 210-250 (EU numbering system).

In some embodiments of the bispecific antibodies represented by structural formula (II) at least one amino acid residue in the hinge region of HCA and HCB may be replaced with at least one cysteine residue, wherein the hinge region of HCA and/or HCB comprises amino acids 210-250 (EU numbering system). the hinge region of HCA and/or HCB consists of amino acids 210-250 (EU numbering system).

In some embodiments of the bispecific antibodies represented by structural formula (II) at least two amino acid residues in the hinge region of HCA and HCB may each be replaced with a cysteine residue, wherein the hinge region of HCA and/or HCB comprises amino acids 210-250 (EU numbering system). the hinge region of HCA and/or HCB consists of amino acids 210-250 (EU numbering system).

### Utilities and Applications

Some embodiments provide a method of treating a patient in need thereof comprising administering a bispecific antibodies as disclosed and described herein to said patient. In some embodiments, the patient may have cancer, an infection, or an immune system disease. In some embodiments, the bispecific antibodies may have anti-tumor, antibiotic, or anti-inflammatory activity.

### Conjugation Methods, Spacers and Linkers Involved

Some embodiments provide a method of making bispecific antibody comprising treating LCA-SH, HCA-SH, LCB-SH, and HCB-SH with X-L-X for a period of time to provide the bispecific antibody, wherein X is halo or —OS(O)₂—R⁶; L is a synthetic linker; and R⁶ is an optionally substituted C₁₋₆ alkyl, optionally substituted aryl or optionally substituted heteroaryl.

Some embodiments provide a method of making bispecific antibody comprising treating LCA-SH, HCA-SH, LCB-SH, and HCB-SH with X-L-X for a period of time to provide the bispecific antibody, wherein X is halo or —OS(O)₂—R⁶; L may be

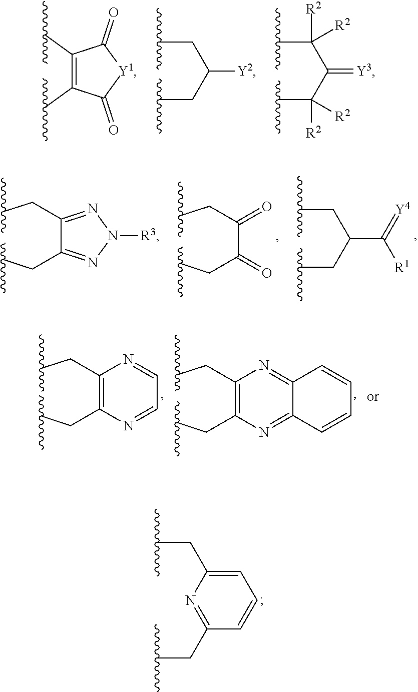

Y¹ may be O (oxygen), NR⁴, —NH—NH—, or —CH═CH—; Y² may be OH or C₁₋₆ alkoxy; Y³ may be O (oxygen), N—OR⁴, or —CF₂—; Y⁴ may be O (oxygen), N—OR⁴, or —CF₂—; R¹ may be H (hydrogen), C₁₋₆ alkyl, aryl, or heteroaryl; R² may be H (hydrogen) or C₁₋₆ alkyl; R³ may be H (hydrogen) or C₁₋₆ alkyl; R⁴ may be H (hydrogen) or C₁₋₆ alkyl; and R⁶ may be a synthetic linker optionally substituted C₁₋₆ alkyl, optionally substituted aryl or optionally substituted heteroaryl.

In some embodiments, the linker may include a 2- to 5-atom bridge. In some embodiments, the linker may include a 2- to 5-carbons. In some embodiments, the linker may include a group including a N (nitrogen) atom. In some embodiments, the method includes a single-step or sequential conjugation approach.

In some embodiments, L¹, L² and L³ may include, but is not limited to,

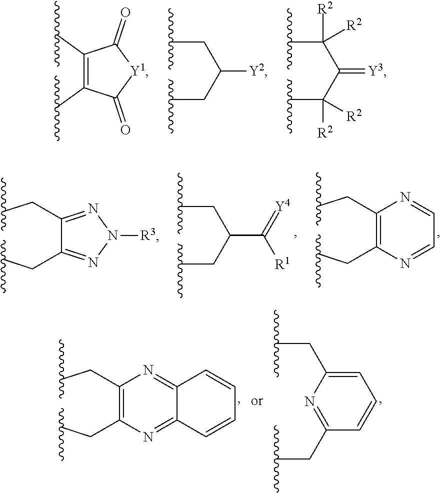

and the like.

As used herein, the term “peptide” refers to a structure including one or more components each individually selected from the group consisting of an amino acid, an amino acid residue, an amino acid analog, and a modified amino acid. The components are typically joined to each other through an amide bond.

As used herein, the term “amino acid” includes naturally occurring amino acids, a molecule having a nitrogen available for forming an amide bond and a carboxylic acid, a molecule of the general formula NH₂—CHR—COOH or the residue within a peptide bearing the parent amino acid, where “R” is one of a number of different side chains. “R” can be a substituent found in naturally occurring amino acids. “R” can also be a substituent referring to one that is not of the naturally occurring amino acids.

As used herein, the term “amino acid residue” refers to the portion of the amino acid which remains after losing a water molecule when it is joined to another amino acid.

As used herein, the term “amino acid analog” refers to a structural derivative of an amino acid parent compound that often differs from it by a single element.

As used herein, the term “modified amino acid” refers to an amino acid bearing an “R” substituent that does not correspond to one of the twenty genetically coded amino acids.

As used herein, the abbreviations for the genetically encoded L-enantiomeric amino acids are conventional and are as follows: The D-amino acids are designated by lower case, e.g. D-proline=p, etc.

TABLE 1A

| Amino Acids | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Certain amino acid residues in the bispecific antibody can be replaced with other amino acid residues without significantly deleteriously affecting, and in many cases even enhancing, the activity. Thus, also contemplated by the preferred embodiments are altered or mutated forms of the bispecific antibody wherein at least one defined amino acid residue in the structure is substituted with another amino acid residue or derivative and/or analog thereof.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are physiologically acceptable solvates including hydrates.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “substituted alkyl” refers to an alkyl group substituted with one or more substituents independently selected from C₁-C₆ alkenyl, C₁-C₆ alkynyl, C₁-C₆ heteroalkyl, C₃-C₇ carbocyclyl (optionally substituted with halo, C1-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ Or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocycyl (optionally substituted with halo, C₁-C₆ alkyl, C₁-C₆ alkoxy, C₁-C₆ haloalkyl, and C₁-C₆ haloalkoxy), 3-10 membered heterocycyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

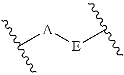

includes the substituent being oriented such that the A is attached at the leftmost attachment point of the molecule as well as the case in which A is attached at the rightmost attachment point of the molecule.

### Pharmaceutical Compositions

In some embodiments, the compounds disclosed herein are used in pharmaceutical compositions. The compounds can be used, for example, in pharmaceutical compositions comprising a pharmaceutically acceptable carrier prepared for storage and subsequent administration. Also, embodiments relate to a pharmaceutically effective amount of the products and compounds disclosed above in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), which is incorporated herein by reference in its entirety. Preservatives, stabilizers, dyes and even flavoring agents can be provided in the pharmaceutical composition. For example, sodium benzoate, ascorbic acid and esters of p-hydroxybenzoic acid can be added as preservatives. In addition, antioxidants and suspending agents can be used.

The compositions can be formulated and used as tablets, capsules, or elixirs for oral administration; suppositories for rectal administration; sterile solutions, suspensions for injectable administration; patches for transdermal administration, and sub-dermal deposits and the like. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (for example, liposomes), can be utilized.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Dragee cores are provided with suitable coatings. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

### Methods of Administration

In an alternative embodiment, the disclosed bispecific antibodies and the disclosed pharmaceutical compositions are administered by a particular method as an anti-cancer, or anti-inflammatory. Such methods include, among others, (a) administration though oral pathways, which administration includes administration in capsule, tablet, granule, spray, syrup, or other such forms; (b) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as (c) administration topically, (d) administration rectally, or (e) administration vaginally, as deemed appropriate by those of skill in the art for bringing the compound of the present embodiment into contact with living tissue; and (f) administration via controlled released formulations, depot formulations, and infusion pump delivery. As further examples of such modes of administration and as further disclosure of modes of administration, disclosed herein are various methods for administration of the disclosed compounds and pharmaceutical compositions including modes of administration through intraocular, intranasal, and intraauricular pathways.

The pharmaceutically effective amount of the compositions that include the described bispecific antibodies required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. In a typical embodiment, bispecific antibodies represented by Formulae I and II can be administered to a patient in need of an anti-cancer agent, until the need is effectively reduced or preferably removed.

In practicing the methods of the embodiment, the products or compositions can be used alone or in combination with one another, or in combination with other therapeutic or diagnostic agents. These products can be utilized in vivo, ordinarily in a mammal, preferably in a human, or in vitro. In employing them in vivo, the products or compositions can be administered to the mammal in a variety of ways, including parenterally, intravenously, subcutaneously, intramuscularly, colonically, rectally, vaginally, nasally or intraperitoneally, employing a variety of dosage forms. Such methods may also be applied to testing chemical activity in vivo.

As will be readily apparent to one skilled in the art, the useful in vivo dosage to be administered and the particular mode of administration will vary depending upon the age, weight and mammalian species treated, the particular compounds employed, and the specific use for which these bispecific antibodies are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired affects and the therapeutic indication. Typically, dosages can be between about 10 mg/kg and 100 mg/kg body weight, preferably between about 100 mg/kg and 10 mg/kg body weight. Alternatively dosages can be based and calculated upon the surface area of the patient, as understood by those of skill in the art. Administration is preferably oral on a daily or twice daily basis.

The exact formulation, route of administration and dosage can be chosen by the individual physician in view of the patient's condition. See for example, Fingl et al., in The Pharmacological Basis of Therapeutics, 1975, which is incorporated herein by reference in its entirety. It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above can be used in veterinary medicine.

Depending on the specific conditions being treated, such agents can be formulated and administered systemically or locally. A variety of techniques for formulation and administration can be found in Remington's Pharmaceutical Sciences, 18th Ed., Mack Publishing Co., Easton, Pa. (1990), which is incorporated herein by reference in its entirety. Suitable administration routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the embodiment can be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the embodiment into dosages suitable for systemic administration is within the scope of the embodiment. With proper choice of carrier and suitable manufacturing practice, the compositions disclosed herein, in particular, those formulated as solutions, can be administered parenterally, such as by intravenous injection. The compounds can be formulated readily using pharmaceutically acceptable carriers well known in the art into dosages suitable for oral administration. Such carriers enable the compounds of the embodiment to be formulated as tablets, pills, capsules, liquids, gels, syrups, slurries, suspensions and the like, for oral ingestion by a patient to be treated.

Agents intended to be administered intracellularly can be administered using techniques well known to those of ordinary skill in the art. For example, such agents can be encapsulated into liposomes, then administered as described above. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. Additionally, due to their hydrophobicity, small organic molecules can be directly administered intracellularly.

Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration can be in the form of tablets, dragees, capsules, or solutions. The pharmaceutical compositions can be manufactured in a manner that is itself known, for example, by means of conventional mixing, dissolving, granulating, dragee-making, levitating, emulsifying, encapsulating, entrapping, or lyophilizing processes.

Bispecific antibodies disclosed herein can be evaluated for efficacy and toxicity using known methods. For example, the toxicology of a particular compound, or of a subset of the compounds, sharing certain chemical moieties, can be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, can be determined using known methods. The efficacy of a particular compound can be established using several art recognized methods, such as in vitro methods, animal models, or human clinical trials. Art-recognized in vitro models exist for nearly every class of condition, including the conditions abated by the bispecific antibodies disclosed herein, including cancer, cardiovascular disease, and various immune dysfunction, and infectious diseases. Similarly, acceptable animal models can be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of bispecific antibodies in humans.

As will be understood by one of skill in the art, “need” is not an absolute term and merely implies that the patient can benefit from the treatment of the anti-cancer agent in use. By “patient” what is meant is an organism that can benefit by the use of an anti-cancer agent.

“Therapeutically effective amount,” “pharmaceutically effective amount,” or similar term, means that amount of drug or pharmaceutical agent that will result in a biological or medical response of a cell, tissue, system, animal, or human that is being sought. In a preferred embodiment, the medical response is one sought by a researcher, veterinarian, medical doctor, or other clinician.

In one embodiment, a described compound, preferably a bispecific antibodies of Formulas I and II, including those as described herein, is considered an effective anti-cancer agent if the compound can influence 10% of the cancer cells, for example. In a more preferred embodiment, the compound is effective if it can influence 10 to 50% of the cancer cells. In an even more preferred embodiment, the compound is effective if it can influence 50-80% of the cancer cells. In an even more preferred embodiment, the compound is effective if it can influence 80-95% of the cancer cells. In an even more preferred embodiment, the compound is effective if it can influence 95-99% of the cancer cells. “Influence” is defined by the mechanism of action for each compound.

### Therapeutic Pharmaceutical Formulations and Administration

The present disclosure provides pharmaceutical formulations containing bispecific antibodies as disclosed and described herein. As used herein, “pharmaceutical formulation” is a sterile composition of a pharmaceutically active drug, namely, at least one bispecific antibodies as disclosed and described herein, that is suitable for parenteral administration (including but not limited to intravenous, intramuscular, subcutaneous, aerosolized, intrapulmonary, intranasal, or intrathecal) to a patient in need thereof and includes only pharmaceutically acceptable excipients, diluents, and other additives deemed safe by the Federal Drug Administration or other foreign national authorities. Pharmaceutical formulations include liquid, e.g., aqueous, solutions that may be directly administered, and lyophilized powders which may be reconstituted into solutions by adding a diluent before administration. Specifically excluded from the scope of the term “pharmaceutical formulation” are compositions for topical administration to patients, compositions for oral ingestion, and compositions for parenteral feeding.

In certain embodiments, the pharmaceutical formulation is a stable pharmaceutical formulation. As used herein, the phrases, “stable pharmaceutical formulation, “stable formulation” or “a pharmaceutical formulation is stable” refers to a pharmaceutical formulation of biologically active proteins that exhibit increased aggregation and/or reduced loss of biological activity of not more than 5% when stored at 2-8° C. for at least 1 month, or 2 months, or 3 months, or 6 months, or 1 year or 2 years compared with a control formula sample. Formulation stability can be easily determined by a person of skill in the art using any number of standard assays, including but not limited to size exclusion HPLC (“SEC-HPLC”), cation-exchange HPLC (CEX-HPLC), Subvisible Particle Detection by Light Obscuration (“HIAC”) and/or visual inspection.

In certain embodiments, the pharmaceutical formulation comprises one or more of the bispecific antibodies as disclosed and described herein.

In certain embodiments, bispecific antibodies as disclosed and described herein is linked to a half-life extending vehicle known in the art. Such vehicles include, but are not limited to, polyethylene glycol, glycogen (e.g., glycosylation of the ABP), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical formulation comprises formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, suitable formulation materials include, but are not limited to, amino acids (such as proline, arginine, lysine, methionine, taurine, glycine, glutamine, or asparagine); antimicrobials; antioxidants (such as ascorbic acid, sodium sulfite or sodium hydrogen-sulfite); buffers (such as borate, bicarbonate, sodium phosphate (“NaOAC”), Tris-HC1, Tris buffer, citrates, phosphate buffer, phosphate-buffered saline (i.e., PBS buffer) or other organic acids); bulking agents (such as mannitol or glycine); chelating agents (such as ethylenediamine tetra acetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, fructose, lactose, mannose, trehelose, or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995).

In certain embodiments, the optimal pharmaceutical formulation will be determined by one skilled in the art depending upon, for example, the intended route of administration, delivery format and desired dosage. See, for example, Remington's Pharmaceutical Sciences, supra. In certain embodiments, such formulations may influence the physical state, stability, rate of in vivo release and rate of in vivo clearance of the antibodies of the invention.

In one aspect, the pharmaceutical formulation comprises high concentrations of a bispecific antibody as disclosed and described herein. In certain embodiments, the pharmaceutical formulation comprises a bispecific antibody as disclosed and described herein in a concentration ranging from about 70 mg/mL to about 250 mg/mL, e.g., about 70 mg/mL, about 80 mg/mL, about 90 mg/mL, about 100 mg/mL, about 100 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, or about 250 mg/mL, and including all values in between. In some embodiments, the concentration of a bispecific antibody as disclosed and described herein ranges from about 100 mg/mL to about 150 mg/mL, e.g., 100 mg/mL, about 100 mg/mL, about 120 mg/mL, about 130 mg/mL, about 140 mg/mL, or about 150 mg/mL. In some embodiments, the concentration of a bispecific antibody as disclosed and described herein ranges from about 140 mg/mL to about 220 mg/mL, e.g., 140 mg/mL, about 150 mg/mL, about 160 mg/mL, about 170 mg/mL, about 180 mg/mL, about 190 mg/mL, about 200 mg/mL, about 210 mg/mL, about 220 mg/mL, or about 250 mg/mL. In one aspect, the pharmaceutical formulation comprises concentrations of a bispecific antibody as disclosed and described herein ranging from about 5 mg/mL to about 65 mg/mL, e.g., about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, or about 65 mg/mL, and including all values in between.

In another aspect, the pharmaceutical formulation comprises at least one buffering agent such as, for example, sodium acetate, sodium chloride, phosphates, phosphate buffered saline (“PBS”), and/or Tris buffer of about pH 7.0-8.5. The buffer serves to maintain a physiologically suitable pH. In addition, the buffer can serve to enhance isotonicity and chemical stability of the pharmaceutical formulation. In certain embodiments, the buffering agent ranges from about 0.05 mM to about 40 mM, e.g., about 0.05 mM, about 0.1 mM, about 0.5 mM, about 1.0 mM, about 5.0 mM, about 10 mM, about 15 mM, about 20 mM, about 30 mM, about 40 mM, about 50 mM, about 60 mM, about 70 mM, about 80 mM, about 90 mM, or about 100 nM buffering agent, inclusive of all values in between. In certain embodiments, the buffering agent is NaOAC. Exemplary pHs of the pharmaceutical formulation include from about 4 to about 6, or from about 4.8 to about 5.8, or from about 5.0 to about 5.2, or about 5, or about 5.2.

In certain embodiments, the pharmaceutical formulation is isotonic with an osmolality ranging from between about 250 to about 350 miliosmol/kg, e.g., about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, or about 350 mOsm/kg, and including all values in between. As used herein, “osmolality” is the measure of the ratio of solutes to volume fluid. In other words, it is the number of molecules and ions (or molecules) per kilogram of a solution. Osmolality may be measured on an analytical instrument called an osmometer, such as Advanced Instruments 2020 Multi-sample Osmometer, Norwood, Mass. The Advanced Instruments 2020 Multi-sample Osmometer measures osmolality by using the Freezing Point Depression method. The higher the osmolytes in a solution, the temperature in which it will freeze drops. Osmolality may also be measured using any other methods and in any other units known in the art such as linear extrapolation.

In still another aspect, the pharmaceutical formulation comprises at least one surfactant including but not limited to Polysorbate-80, Polysorbate-60, Polysorbate-40, and Polysorbate-20. In certain embodiments, the pharmaceutical formulation comprises a surfactant at a concentration that ranges from about 0.004% to about 10% weight per volume (“w/v”) of the formulation, e.g., about 0.004%, about 0.005%, about 0.006%, about 0.007%, about 0.008%, about 0.009%, about 0.01%, about 0.05%, about 0.1%, about 0.5%, about 1%, about 5%, or about 10% surfactant w/v of the formulation. In certain embodiments, the pharmaceutical formulation comprises polysorbate 80 at a concentration that ranges from about 0.004% to about 0.1% w/v of the formulation. In certain embodiments, the pharmaceutical formulation comprises polysorbate 20 at a concentration that ranges from about 0.004% to about 0.1% w/v of the formulation.

In certain embodiments, the pharmaceutical formulation comprises at least one stabilizing agent, such as a polyhydroxy hydrocarbon (including but not limited to sorbitol, mannitol, glycerol and dulcitol) and/or a disaccharide (including but not limited to sucrose, lactose, maltose and threhalose) and/or an amino acid (including but not limited to proline, arginine, lysine, methionine, and taurine) and or benzyl alcohol; the total of said polyhydroxy hydrocarbon and/or disaccharide and/or amino acid and/or benzyl alcohol being about 0.5% to about 10% w/v of the formulation. In certain embodiments, the pharmaceutical formulation comprises a stabilizing agent at a concentration of about 1%, about 2%, about 3%, about 4%, about 5%, about 6%, about 7%, about 8%, about 9% or about 10% sucrose. In certain embodiments, the pharmaceutical formulation comprises a stabilizing agent at a concentration of about 5% sucrose. In certain embodiments, the pharmaceutical formulation comprises a stabilizing agent at a concentration of about 1%, about 2%, about 3%, about 4%, about 5%, about 6%, about 7%, about 8%, about 9% or about 10% sorbital. In certain embodiments, the pharmaceutical formulation comprises a stabilizing agent at a concentration of about 9% sorbital. In certain embodiments, the pharmaceutical formulation comprises a stabilizing agent at a concentration of about 1%, about 2%, about 3%, about 4%, about 5% proline, arginine, lysine, methionine, and/or taurine. In certain embodiments, the pharmaceutical formulation comprises a stabilizing agent at a concentration of between about 2-3% proline. In certain embodiments, the pharmaceutical formulation comprises a stabilizing agent at a concentration of about 1%, about 2%, about 3%, about 4%, about 5% benzyl alcohol. In certain embodiments, the pharmaceutical formulation comprises a stabilizing agent at a concentration of between about 1-2% benzyl alcohol.

In one aspect, the pharmaceutical formulation has a viscosity level of less than about 30 centipoise (cP) as measured at room temperature (i.e., 25 C). As used herein, “viscosity” is a fluid's resistance to flow, and may be measured in units of centipoise (cP) or milliPascal-second (mPa-s), where 1 cP=1 mPa-s, at a given shear rate. Viscosity may be measured by using a viscometer, e.g., Brookfield Engineering Dial Reading Viscometer, model LVT. Viscosity may also be measured using any other methods and in any other units known in the art (e.g., absolute, kinematic or dynamic viscosity or absolute viscosity). In certain embodiments, the pharmaceutical formulation has a viscosity level of less than about 25 cP, about 20 cP, about 18 cP, about 15 cP, about 12 cP, about 10 cP; about 8 cP, about 6 cP, about 4 cP; about 2 cP; or about 1 cP.

In one aspect, the pharmaceutical formulation is stable as measured by at least one stability assay known to one of skill in the art, such as assays that examine the biophysical or biochemical characteristics of biologically active proteins over time. As mentioned above, a stable pharmaceutical formulation of the present invention is a pharmaceutical formulation of biologically active proteins that exhibits increased aggregation and/or reduced loss of biological activity of not more than 5% when stored at 2-8° C. for at least 1 month, or 2 months, or 3 months, or 6 months, or 1 year or 2 years compared with a control formula sample. In certain embodiments, the pharmaceutical formulation stability is measured using size exclusion HPLC (“SEC-HPLC”). SEC-HPLC separates proteins based on differences in their hydrodynamic volumes. Molecules with larger hydrodynamic proteins volumes elute earlier than molecules with smaller volumes. In the case of SEC-HPLC, a stable pharmaceutical formulation should exhibit no more than about a 5% increase in high molecular weight species as compared to a control sample. In certain other embodiments, the pharmaceutical formulation should exhibit no more than about a 4%, no more than about a 3%, no more than about a 2%, no more than about a 1%, no more than about a 0.5% increase in high molecular weight specifies as compared to a control sample.

In certain embodiments, the pharmaceutical formulation stability is measured using cation-exchange HPLC (CEX-HPLC). CEX-HPLC separates proteins based on differences in their surface charge. At a set pH, charged isoforms of an bispecific antibody are separated on a cation-exchange column and eluted using a salt gradient. The eluent is monitored by UV absorbance. The charged isoform distribution is evaluated by determining the peak area of each isoform as a percent of the total peak area. In the case of CEX-HPLC, a stable pharmaceutical formulation should exhibit no more than about a 5% decrease in the main isoform peak as compared to a control sample. In certain other embodiments, a stable pharmaceutical formulation should exhibit no more than about a 3% to about a 5% decrease in the main isoform peak as compared to a control sample. In certain embodiments, the pharmaceutical formulation should exhibit no more than about a 4% decrease, no more than about a 3% decrease, no more than about a 2% decrease, no more than about a 1% decrease, no more than about a 0.5% decrease in the main isoform peak as compared to a control sample.

In certain embodiments, the pharmaceutical formulation stability is measured using Subvisible Particle Detection by Light Obscuration (“HIAC”). An electronic, liquid-borne particle-counting system (HIAC/Royco 9703 or equivalent) containing a light-obscuration sensor (HIAC/Royco HRLD-150 or equivalent) with a liquid sampler quantifies the number of particles and their size range in a given test sample. When particles in a liquid pass between the light source and the detector they diminish or “obscure” the beam of light that falls on the detector. When the concentration of particles lies within the normal range of the sensor, these particles are detected one-by-one. The passage of each particle through the detection zone reduces the incident light on the photo-detector and the voltage output of the photo-detector is momentarily reduced. The changes in the voltage register as electrical pulses that are converted by the instrument into the number of particles present. The method is non-specific and measures particles regardless of their origin. Particle sizes monitored are generally 10 um, and 25 um. In the case of HIAC, a stable pharmaceutical formulation should exhibit no more than 6000 10 μm particles per container (or unit), as compared to a control sample. In certain embodiments, a stable pharmaceutical formulation should exhibit no more than 5000, no more than 4000, no more than 3000, no more than 2000, no more than 1000, 10 μm particles per container (or unit) as compared to a control sample. In still other embodiments, a stable pharmaceutical formulation should exhibit no more than 600 25 μm particles per container (or unit) as compared to a control sample. In certain embodiments, a stable pharmaceutical formulation should exhibit no more than 500, no more than 400, no more than 300, no more than 200, no more than 100, no more than 50 25 μm particles per container (or unit) as compared to a control sample.

In certain embodiments, the pharmaceutical formulation stability is measured using visual assessment. Visual assessment is a qualitative method used to describe the visible physical characteristics of a sample. The sample is viewed against a black and/or white background of an inspection booth, depending on the characteristic being evaluated (e.g., color, clarity, presence of particles or foreign matter). Samples are also viewed against an opalescent reference standard and color reference standards. In the case of visual assessment, a stable pharmaceutical formulation should exhibit no significant change in color, clarity, presence of particles or foreign matter as compared to a control sample.

One aspect of the present invention is a pharmaceutical formulation which comprises: (i) about 70 mg/mL to about 250 mg/mL of a bispecific antibody as disclosed and described herein; (ii) about 0.05 mM to about 40 mM of a buffer such as sodium acetate (“NaOAC”) serves as a buffering agent; (iii) about 1% to about 5% proline, arginine, lysine, methionine, or taurine (also known as 2-aminoethanesulfonic acid) and/or 0.5% to about 5% benzyl alcohol which serves as a stabilizing agent; and (iv) about 0.004% to about 10% w/v of the formulation of a non-ionic surfactant (including but not limited to Polysorbate-80, Polysorbate-60, Polysorbate-40, and Polysorbate-20); wherein said formulation has a pH in the range of about 4.0 to 6.0. In certain other embodiments, pharmaceutical formulations of this invention comprise (i) at least about 70 mg/mL, about 100 mg/mL, about 120 mg/mL, about 140 mg/mL, about 150 mg/mL, about 160 mg/mL, about 170 mg/mL, about 180 mg/mL, about 190 mg/mL, about 200 mg/mL of a bispecific antibody as disclosed and described herein; (ii) about 10 mM NAOAC; (iii) about 0.01% polysorbate 80; and (iv) between about 2%-3% proline (or about 250 mM to about 270 mM proline), wherein the formulation has a pH of about 5. In certain other embodiments, pharmaceutical formulations of this invention comprise (i) at least about 70 mg/mL, about 100 mg/mL, about 120 mg/mL, about 140 mg/mL of a bispecific antibody as disclosed and described herein; (ii) about 10 mM NAOAC; (iii) about 0.01% polysorbate 80; and (iv) between about 2%-3% proline (or about 250 mM to about 270 mM proline), wherein the formulation has a pH of about 5. In certain other embodiments, pharmaceutical formulations of this invention comprise (i) at least about 150 mg/mL, about 160 mg/mL, about 170 mg/mL, about 180 mg/mL, about 190 mg/mL, about 200 mg/mL of a bispecific antibody as disclosed and described herein; (ii) about 10 mM NAOAC; (iii) about 0.01% polysorbate 80; and (iv) between about 2%-3% proline (or about 250 mM to about 270 mM proline), wherein the formulation has a pH of about 5.

One aspect of the present invention is a pharmaceutical formulation which comprises (i) at least about 70 mg/mL to about 250 mg/mL of a bispecific antibody as disclosed and described herein; (ii) about 5 mM to about 20 mM of a buffer, such as NAOAC; (iii) about 1% to about 10% w/v of the formulation comprises a polyhydroxy hydrocarbon such as sorbitol, or a disaccharide such as sucrose; and (iv) about 0.004% to about 10% w/v of the formulation of a surfactant, such as polysorbate 20 or polysorbate 80; wherein said formulation has a pH in the range of about 4.8 to 5.8; and wherein the pharmaceutical formulation optionally comprises about 80 mM to about 300 mM proline, arginine, lysine, methionine, or taurine and/or 0.5% to about 5% benzyl alcohol which serves to reduce viscosity. In certain other embodiments, pharmaceutical formulations of this invention comprise (i) at least about 70 mg/ml to about 250 mg/ml of a bispecific antibody as disclosed and described herein; (ii) about 10 mM NAOAC; (iii) about 9% sucrose; and (iv) about 0.004% polysorbate 20, wherein the formulation has a pH of about 5.2. In certain other embodiments, pharmaceutical formulations of this invention comprise (i) at least about 70 mg/mL, about 100 mg/mL, about 120 mg/mL, about 140 mg/mL, about 160 mg/mL, about 180 mg/mL, about 200 mg/mL of a bispecific antibody as disclosed and described herein; (ii) about 15 mM NAOAC; (iii) about 9% sucrose; and (iv) about 0.01% polysorbate 20, wherein the formulation has a pH of about 5.2. In certain other embodiments, pharmaceutical formulations of this invention comprise (i) at least about 70 mg/mL, about 100 mg/mL, about 120 mg/mL, about 140 mg/mL, about 160 mg/mL, about 180 mg/mL, about 200 mg/mL of a bispecific antibody as disclosed and described herein; (ii) about 20 mM NAOAC; (iii) about 9% sucrose; and (iv) about 0.01% polysorbate 20, wherein the formulation has a pH of about 5.2. In certain other embodiments, pharmaceutical formulations of this invention comprise (i) at least about 70 mg/mL, about 100 mg/mL, about 120 mg/mL, about 140 mg/mL, about 160 mg/mL, about 180 mg/mL, about 200 mg/mL of a bispecific antibody as disclosed and described herein; (ii) about 10 mM NAOAC; (iii) about 9% sucrose; (iv) about 0.01% polysorbate 80; and (v) about 250 mM proline, wherein the formulation has a pH of about 5.

### Methods of Treatment

Some embodiments include methods of treating cancer by administering a compound of any one of Formula I and/or II to a subject in need of cancer therapy. Non-limiting cancers that can be treated using the compounds described herein include bladder cancer, breast cancer, colon cancer, rectal cancer, endometrial cancer, kidney cancer, lung cancer, melanoma, non-Hodgkin lymphoma, glioblastoma, pancreatic cancer, prostate cancer, ovarian cancer and thyroid cancer.

Some embodiments include the treatment of cancer including, but not limited to a carcinoma, a sarcoma, a lymphoma, a leukemia, and a blastoma. Non-limiting cancers that can be treated using the compounds described herein include bladder cancer, breast cancer, colon cancer, rectal cancer, endometrial cancer, kidney cancer, lung cancer, melanoma, non-Hodgkin lymphoma, leukemia, glioblastoma, pancreatic cancer, prostate cancer, ovarian cancer and thyroid cancer.

Some embodiments include the treatment of cancer including, but not limited to a carcinoma, a sarcoma, a lymphoma, a leukemia, and a blastoma. Non-limiting cancers that can be treated using the compounds described herein include bladder cancer, breast cancer, colon cancer, rectal cancer, endometrial cancer, kidney cancer, lung cancer, melanoma, non-Hodgkin lymphoma, leukemia, glioblastoma, pancreatic cancer, prostate cancer, ovarian cancer and thyroid cancer.

Some embodiments provide a method of treating a cancer comprising administering a compound of Formula I or II, or a pharmaceutically acceptable salt thereof to a subject in need thereof. In some embodiments, the cancer is pancreatic cancer. In some embodiments, the cancer is ovarian cancer. In some embodiments, the cancer is prostate cancer.

Some embodiments provide a method of treating melanoma, comprising administering a compound of Formula I or II, or a pharmaceutically acceptable salt thereof to a subject in need thereof. Some embodiments provide a method of treating multiple myeloma, comprising administering a compound of Formula I or II, or a pharmaceutically acceptable salt thereof to a subject in need thereof.

### Methods of Preparation

The bispecific antibodies disclosed herein may be synthesized by methods described below, or by modification of these methods. Ways of modifying the methodology include, among others, temperature, solvent, reagents etc., known to those skilled in the art. In general, during any of the processes for preparation of the bispecific antibodies disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Some exemplary synthetic methods for preparing the present compounds are illustrated in the Schemes 1 and 2 below.

Scheme 1: Preparation of LCA-SH, HCA-SH, LCB-SH and HCB-SH:

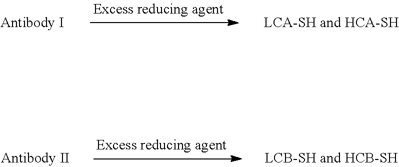

As shown in Scheme 1, LCA-SH and HCA-SH, which are key building blocks in the synthesis of bispecific antibody represented by structural formula (I) can be prepared by treating Antibody I with excess reducing agent in a solvent, and LCB-SH and HCB-SH, which are key building blocks in the synthesis of bispecific antibody represented by structural formula (I) can be prepared by treating Antibody II with excess reducing agent in a solvent. For example, Antibody I may be treated with 4-100 equiv. tris(2-carboxyethyl)phosphine (TECP) or dithiothreitol (DTT) in a pH buffered solution (4.0-9.0) under slight stirring or agitation. The initial mixture may be kept at 0-40° C. for 0.5-12 h. The initial mixtures of LCA-SH and HCA-SH and LCB-SH and HCB-SH maybe further mixed in about 1:1 molar ratio, and then the excess reducing agent removed by buffer exchange via ultra filtration or dialysis. The mixture of LCA-SH, HCA-SH and LCB-SH and HCB-SH may be kept 0-40° C. for 0-12 h before subjected to the next coupling reaction step.

Scheme 2: Preparation of Bispecific Antibody Represented by Structural Formula (I):

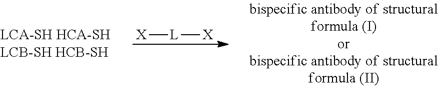

As shown in Scheme 2, of bispecific antibody represented by structural formula (I) or of bispecific antibody represented by structural formula (II) can be prepared by treating the mixture of LCA-SH, HCA-SH and LCB-SH and HCB-SH with 1-200 equiv of bifunctional linker reagent such as epihalohydrin, or X-L-X where X may be halo, or —OS(O)₂—R⁶, L may be a synthetic linker, and R⁶ may be an optionally substituted C₁₋₆ alkyl, optionally substituted aryl or optionally substituted heteroaryl. The resulting mixture may be kept at 0-40° C. for 0.5-12 h under slight stirring or agitation. The progress of product formation may be monitored by HIC-HPLC or RP-HPLC and SDS-PAGE, and then the excess linker may be removed from the solution by ultrafiltration or HIC-HPLC or IEC. The formed bispecific antibody represented by structural formula (I) or of bispecific antibody represented by structural formula (II) may be further purified and fully characterized for use.

In some embodiments, the bifunctional linker reagent may be

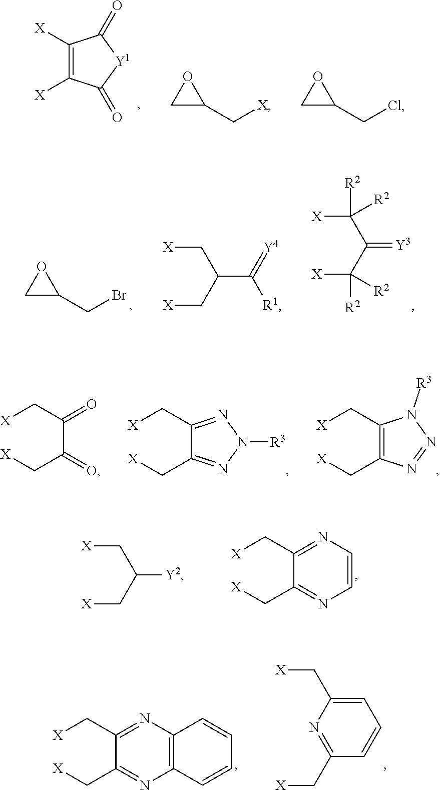

where X may be halo, or —OS(O)₂—R⁶, R⁶ may be an optionally substituted C₁₋₆ alkyl, optionally substituted aryl or optionally substituted heteroaryl, Y¹ may be O (oxygen), NR⁴, —NH—NH—, or —CH═CH—, Y² may be OH or C₁₋₆ alkoxy, Y³ may be O (oxygen), N—OR⁴, or —CF₂—, Y⁴ may be O (oxygen), N—OR⁴, or —CF₂—, R¹ may be H (hydrogen), C₁₋₆ alkyl, aryl, or heteroaryl, R² may be H (hydrogen) or C₁₋₆ alkyl, R³ may be H (hydrogen) or C₁₋₆ alkyl, and R⁴ may be H (hydrogen) or C₁₋₆ alkyl. In some embodiments, the bifunctional linker reagent may be

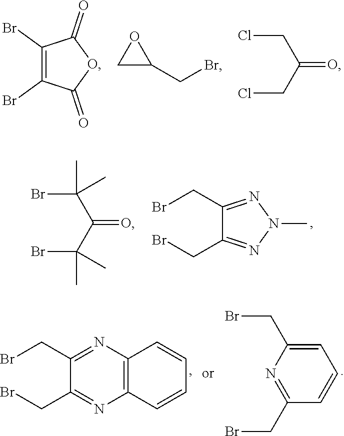

In some embodiments, antibody I may be Adalimumab, Bevacizumab, Anti-IL17, Anti-PDGF, Anti-Ang2, Anti-HGF, Anti-CD3, Anti-CD20, anti-CLL1, Anti-mesothelin. Anti-CD47, Anti-5T4, Anti-Trop2 or Anti-GPC3 and antibody II may be Adalimumab, Bevacizumab, Anti-IL17, Anti-PDGF, Anti-Ang2, Anti-HGF, Anti-CD3, Anti-CD20, anti-CLL1, Anti-mesothelin. Anti-CD47, Anti-5T4, Anti-Trop2 or Anti-GPC3 where wherein antibody I and antibody II are not the same antibody.

Although the disclosure has been described with reference to embodiments and examples, it should be understood that numerous and various modifications can be made without departing from the spirit of the invention. Accordingly, the invention is limited only by the following claims.

### EXAMPLES

The following examples are set forth merely to assist in understanding the embodiments and should not be construed as limiting the embodiments described and claimed herein in any way. Variations of the invention, including the substitution of all equivalents now known or later developed, which would be within the purview of those skilled in the art, and changes in formulation or minor changes in experimental design, are to be considered to fall within the scope of the invention incorporated herein.

### Example 1

### General Procedure A—Synthesis of Mixture of LCA-SH and HCA-SH and LCB-SH and HCB-SH

To a separate solution of 2-50 mg/mL of antibody-1 or antibody-2 in a buffer at pH 4.0-9.0, 4-100 equiv. of the reducing agent TECP is added, respectively, under slight stirring or agitation. The reaction is kept at 0-40° C. for 0.5-12 h. The resultant reaction solutions of LCA-SH and HCA-SH from antibody-1 and LCB-SH and HCB-SH from antibody-2 are mixed in about 1:1 molar ratio, and then the excess reducing agent is removed from the solution by buffer exchange via ultrafiltration or dialysis. The reduced antibody mixture of LCA-SH and HCA-SH and LCB-SH and HCB-SH is kept 0-40° C. for 0-12 h before subjected to the next coupling reaction step.

### General Procedure B—Synthesis of Mixture of LCA-SH and HCA-SH and LCB-SH and HCB-SH

To a solution of 4-100 mg/mL of antibody-1 and antibody-2 in a buffer at pH 4.0-9.0, 4-100 equiv. of the reducing agent TECP is added, respectively, under slight stirring or agitation where antibody-1 and antibody-2 are in about 1:1 molar ratio. The reaction is kept at 0-40° C. for 0.5-12 h. Subsequently, the excess reducing agent is removed from the solution by buffer exchange via ultrafiltration or dialysis. The reduced antibody mixture of LCA-SH and HCA-SH and LCB-SH and HCB-SH is kept 0-40° C. for 0-12 h before subjected to the next coupling reaction step.

The resulting mixtures are shown in Tables 1-11 below.

TABLE 1

| antibody | HCA-SH | LCA-SH | HCB-SH | LCB-SH |
|---|---|---|---|---|
| Adalimumab | SEQ ID | SEQ ID | | |
| | NO: 1 | NO: 2 | | |
| Anti-IL17 | | | SEQ ID NO: 5 | SEQ ID NO: 6 |
| Anti-IL17 | | | SEQ ID NO: 7 | SEQ ID NO: 8 |

TABLE 2

| antibody | HCA-SH | LCA-SH | HCB-SH | LCB-SH |
|---|---|---|---|---|
| Bevacizumab | SEQ ID | SEQ ID | | |
| | NO: 3 | NO: 4 | | |
| Anti-IL17 | | | SEQ ID NO: 5 | SEQ ID NO: 6 |
| Anti-IL17 | | | SEQ ID NO: 7 | SEQ ID NO: 8 |

TABLE 3

| antibody | HCA-SH | LCA-SH | HCB-SH | LCB-SH |
|---|---|---|---|---|
| Bevacizumab | SEQ ID | SEQ ID | | |
| | NO: 3 | NO: 4 | | |
| Anti-Ang2 | | | SEQ ID NO: 11 | SEQ ID |
| | | | | NO: 12 |

TABLE 4

| antibody | HCA-SH | LCA-SH | HCB-SH | LCB-SH |
|---|---|---|---|---|
| Anti-HGF | SEQ ID | SEQ ID | | |
| | NO: 13 | NO: 14 | | |
| Bevacizumab | | | SEQ ID NO: 3 | SEQ ID NO: 4 |

TABLE 5

| antibody | HCA-SH | LCA-SH | HCB-SH | LCB-SH |
|---|---|---|---|---|
| Anti-CD3 | SEQ ID NO: | SEQ ID | | |
| | 15 | NO: 16 | | |
| Anti-CD20 | | | SEQ ID NO: 17 | SEQ ID NO: |
| | | | | 18 |

TABLE 6

| antibody | HCA-SH | LCA-SH | HCB-SH | LCB-SH |
|---|---|---|---|---|
| Anti-CD3 | SEQ ID NO: 15 | SEQ ID NO: 16 | | |
| Anti-5T4 | | | SEQ ID | SEQ ID |
| | | | NO: 25 | NO: 26 |

TABLE 7

| antibody | HCA-SH | LCA-SH | HCB-SH | LCB-SH |
|---|---|---|---|---|
| Anti-mesothelin | SEQ ID | SEQ ID | | |
| | NO: 21 | NO: 22 | | |
| Anti-CD3 | | | SEQ ID NO: 15 | SEQ ID |
| | | | | NO: 16 |

TABLE 8

| antibody | HCA-SH | LCA-SH | HCB-SH | LCB-SH |
|---|---|---|---|---|
| Anti-CD47 | SEQ ID NO: | SEQ ID NO: 24 | | |
| | 23 | | | |
| Anti-5T4 | | | SEQ ID NO: | SEQ ID |
| | | | 25 | NO: 26 |

TABLE 9

| antibody | HCA-SH | LCA-SH | HCB-SH | LCB-SH |
|---|---|---|---|---|
| Anti-CD47 | SEQ ID NO: | SEQ ID | | |
| | 23 | NO: 24 | | |
| Anti-GPC3 | | | SEQ ID NO: 29 | SEQ ID NO: |
| | | | | 30 |

TABLE 10

| antibody | HCA-SH | LCA-SH | HCB-SH | LCB-SH |
|---|---|---|---|---|
| Anti-CD47 | SEQ ID NO: | SEQ ID NO: 24 | | |
| | 23 | | | |
| Anti- | | | SEQ ID | SEQ ID |
| mesothelin | | | NO: 21 | NO: 22 |

TABLE 11

| antibody | HCA-SH | LCA-SH | HCB-SH | LCB-SH |
|---|---|---|---|---|
| Anti-CD47 | SEQ ID NO: | SEQ ID NO: 24 | | |
| | 23 | | | |
| Anti-Trop2 | | | SEQ ID | SEQ ID |
| | | | NO: 27 | NO: 28 |

### Example 2

### General Procedure B—Synthesis of Bispecific Antibody

To the obtained mixture of Tables 1-11, 1-200 equiv. of bifunctional linker epihalohydrin or X-L-X is added. The resulting mixture is kept at 0-40° C. for 0.5-12 h under slight stirring or agitation. The progress of the reaction is monitored by HIC-HPLC or RP-HPLC and SDS-PAGE, and then the excess linker is removed from the solution by ultra filtration or HIC-HPLC or IEC. The formed bispecific antibody is further purified and characterized.

The bispecific antibodies synthesized from the mixture of Tables 1-11 are shown in Tables 12-22 below where the portions of the bispecific antibodies are defined according to Formula (II). The bifunctional linker reagent is

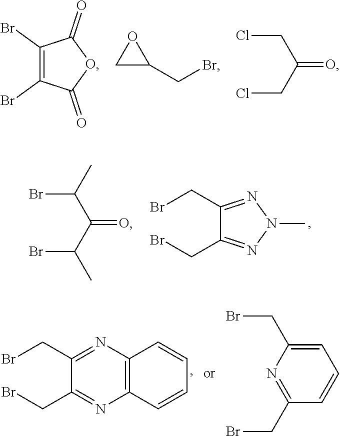

and provides the corresponding L¹, L² and L³ portion of the bispecific antibodies after loss of halogens, or epoxide opening and loss halogen.

TABLE 12
(Corresponds to Table 1 (Adalimumab-Anti-IL17))

| L¹, L² and L³ | HCA-S- | LCA-S- | HCB-S- | LCB-S- |
|---|---|---|---|---|
| 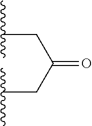 [SMILES: *CC(=O)C*] | SEQ. ID #1 | SEQ. ID #2 | SEQ. ID #5, SEQ. ID #7 | SEQ. ID #6, SEQ. ID #8 |

TABLE 13
(Corresponds to Table 2 (Bevacizumab-Anti-IL17))

| L¹, L² and L³ | HCA-S- | LCA-S- | HCB-S- | LCB-S- |
|---|---|---|---|---|
| 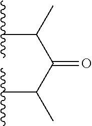 [SMILES: *C(C)C(=O)C(*)C] | SEQ. ID #3 | SEQ. ID #4 | SEQ. ID #5, SEQ. ID #7 | SEQ. ID #6, SEQ. ID #8 |

TABLE 14
(Corresponds to Table 3 (Bevacizumab-Anti-Ang2))

| L¹, L² and L³ | HCA-S- | LCA-S- | HCB-S- | LCB-S- |
|---|---|---|---|---|
| 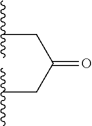 [SMILES: *CC(=O)C*] | SEQ. ID #3 | SEQ. ID #4 | SEQ. ID #11 | SEQ. ID #12 |

TABLE 15
(Corresponds to Table 4 (Anti-HGF-Bevacizumab))

| L¹, L² and L³ | HCA-S- | LCA-S- | HCB-S- | LCB-S- |
|---|---|---|---|---|
| 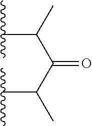 [SMILES: *C(C)C(=O)C(*)C] | SEQ. ID #13 | SEQ. ID #14 | SEQ. ID #3 | SEQ. ID #4 |

TABLE 16
(Corresponds to Table 5 (Anti-CD3-Anti-CD20))

| L¹, L² and L³ | HCA-S- | LCA-S- | HCB-S- | LCB-S- |
|---|---|---|---|---|
| 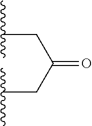 [SMILES: *CC(=O)C*] | SEQ. ID #15 | SEQ. ID #16 | SEQ. ID #17 | SEQ. ID #18 |

TABLE 17
(Corresponds to Table 6 (Anti-CD3-Anti-5T4))

| L¹, L² and L³ | HCA-S- | LCA-S- | HCB-S- | LCB-S- |
|---|---|---|---|---|
| 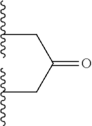 [SMILES: *CC(=O)C*] | SEQ. ID #15 | SEQ. ID #16 | SEQ. ID #25 | SEQ. ID #26 |

TABLE 18
(Corresponds to Table 7 (Anti-mesothelin-Anti-CD3))

| L¹, L² and L³ | HCA-S- | LCA-S- | HCB-S- | LCB-S- |
|---|---|---|---|---|
| 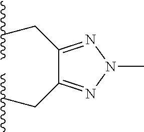 [SMILES: *Cc1nn(C)nc1C*] | SEQ. ID #21 | SEQ. ID #22 | SEQ. ID #15 | SEQ. ID #16 |

TABLE 19
(Corresponds to Table 8 (Anti-CD47-Anti-5T4))

| L¹, L² and L³ | HCA-S- | LCA-S- | HCB-S- | LCB-S- |
|---|---|---|---|---|
| 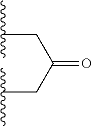 [SMILES: *CC(=O)C*] | SEQ. ID #23 | SEQ. ID #24 | SEQ. ID #25 | SEQ. ID #26 |

TABLE 20
(Corresponds to Table 9 (Anti-CD47-Anti-GPC3))

| L¹, L² and L³ | HCA-S- | LCA-S- | HCB-S- | LCB-S- |
|---|---|---|---|---|
| 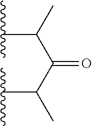 [SMILES: *C(C)C(=O)C(*)C] | SEQ. ID #23 | SEQ. ID #24 | SEQ. ID #29 | SEQ. ID #30 |

TABLE 21
(Corresponds to Table 10 (Anti-CD47-Anti-mesothelin))

| L¹, L² and L³ | HCA-S- | LCA-S- | HCB-S- | LCB-S- |
|---|---|---|---|---|
| 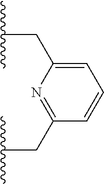 [SMILES: *Cc1cccc(C*)n1] | SEQ. ID #23 | SEQ. ID #24 | SEQ. ID #21 | SEQ. ID #22 |

TABLE 22
(Corresponds to Table 11 (Anti-CD47-Anti-Trop2))

| L¹, L² and L³ | HCA-S- | LCA-S- | HCB-S- | LCB-S- |
|---|---|---|---|---|
| 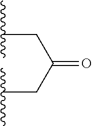 [SMILES: *CC(=O)C*] | SEQ. ID #23 | SEQ. ID #24 | SEQ. ID #27 | SEQ. ID #28 |

### Example 3

FIG. 1 provides a bispecific antibody synthesized using six different bifunctional linker reagents (1-6).

### Example 4

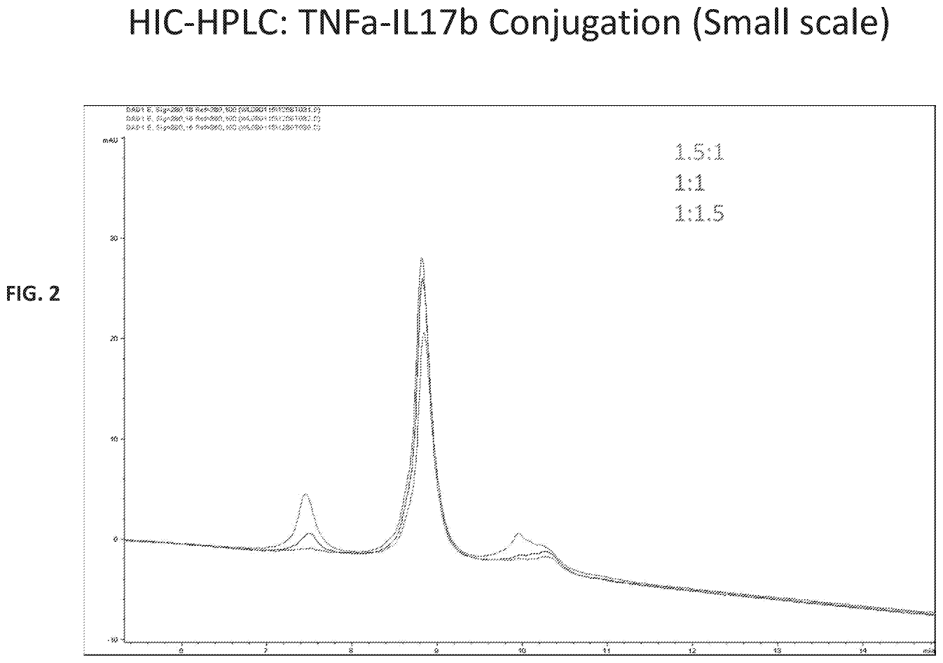

FIG. 2 provides the HIC-HPLC of the bispecific antibody of Table 12 synthesized according to General procedure B using 1,3-dichloroacetone as X-L-X. The mixture of LCA-SH and HCA-SH from reduction of Adalimumab antibody and the mixture of LCB-SH and HCB-SH from reduction of Anti-IL17 antibody were reacted together in three different ratios, 1.5:1, 1:1 and 1:1.5.

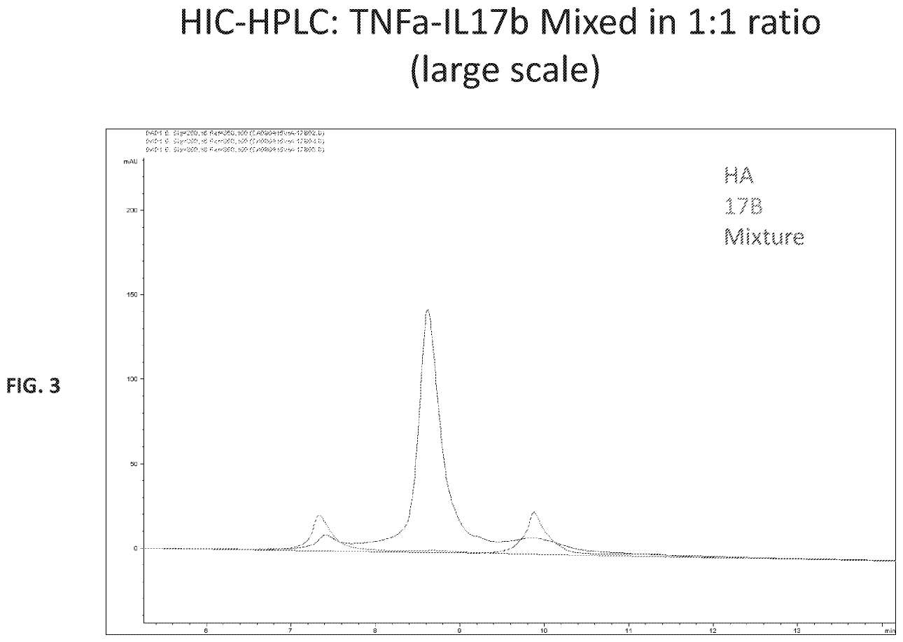

FIG. 3 provides the HIC-HPLC of a mixture of the bispecific antibody of Table 12 with Adalimumab antibody and Anti-IL17 antibody demonstrating that the bispecific antibody of Table 12 is not Adalimumab antibody or Anti-IL17 antibody. The ratio of the mixture of LCA-SH and HCA-SH from reduction of Adalimumab antibody and the mixture of LCB-SH and HCB-SH from reduction of Anti-IL17 antibody that were reacted together according to General procedure B is 1:1 using 1,3-dichloroacetone as X-L-X.

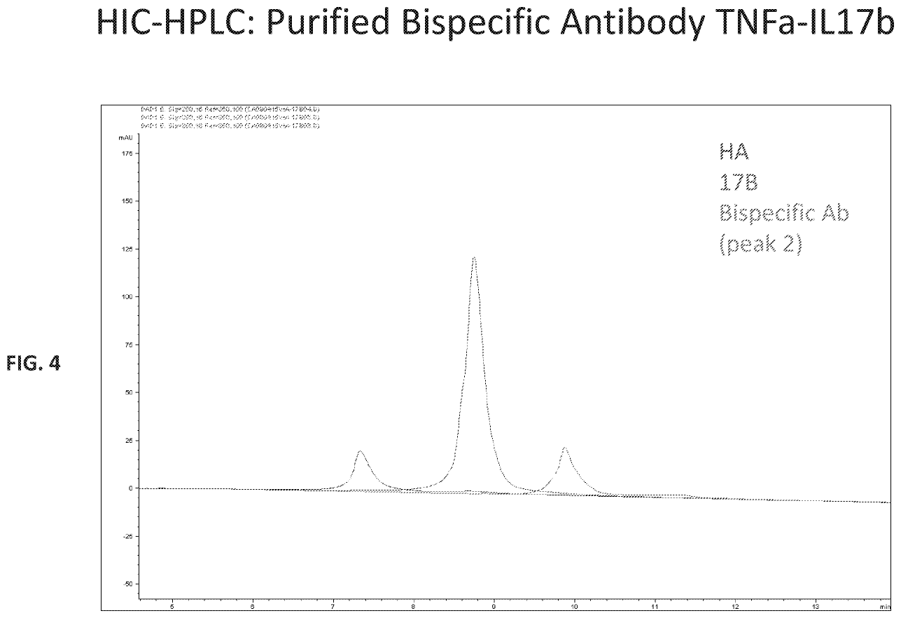

FIG. 4 provides the HIC-HPLC of a mixture of purified bispecific antibody of Table 12 (1,3-dichloroacetone as the linker reagent of General procedure B, X-L-X) with Adalimumab antibody and Anti-IL17 antibody demonstrating that the bispecific antibody of Table 12 is not Adalimumab antibody or Anti-IL17 antibody using 1,3-dichloroacetone as the linker reagent.

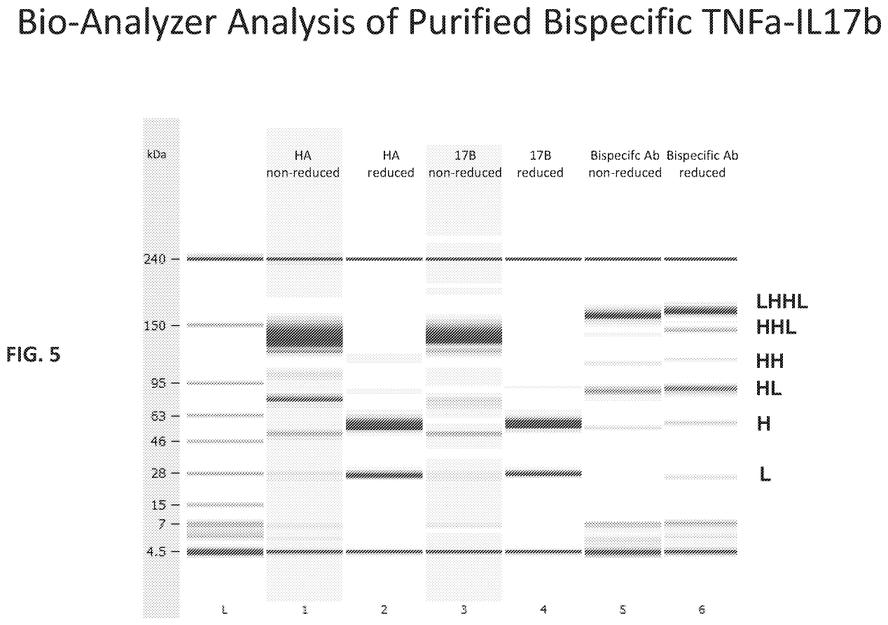

FIG. 5 provides Bio-Analyzer Analysis comparing bispecific antibody of Table 12 (1,3-dichloroacetone as the linker reagent of General procedure B, X-L-X) with Adalimumab antibody and Anti-IL17 antibody, each in reduced and non-reduced form.

### Example 5

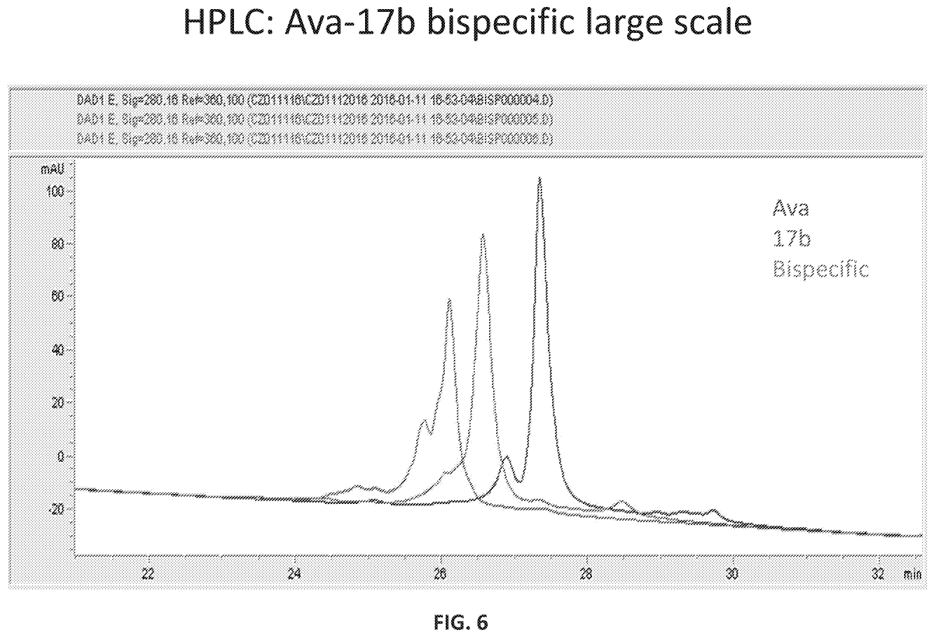

FIG. 6 provides the HPLC of the bispecific antibody of Table 13 (2,4-dibromo-3-pentanone or 1,3-dichloroacetone as the linker reagent of General procedure B, X-L-X) synthesized according to General procedure B in a mixture with Bevacizumab antibody and Anti-IL17 antibody demonstrating that the bispecific antibody of Table 13 is not Bevacizumab antibody or Anti-IL17 antibody. The mixture of LCA-SH and HCA-SH from Adalimumab and the mixture of LCB-SH and HCB-SH from Anti-IL17 were reacted together in three different ratios, 1.5:1, 1:1 and 1:1.5.

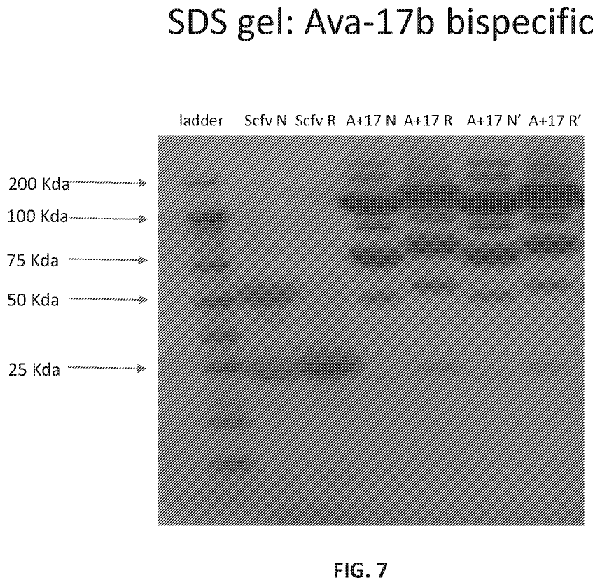

FIG. 7 provides SDS gel of the bispecific antibody of Table 13A (2,4-dibromo-3-pentanone or 1,3-dichloroacetone as the linker reagent of General procedure B, X-L-X) in reduced and non-reduced form.

### Example 6

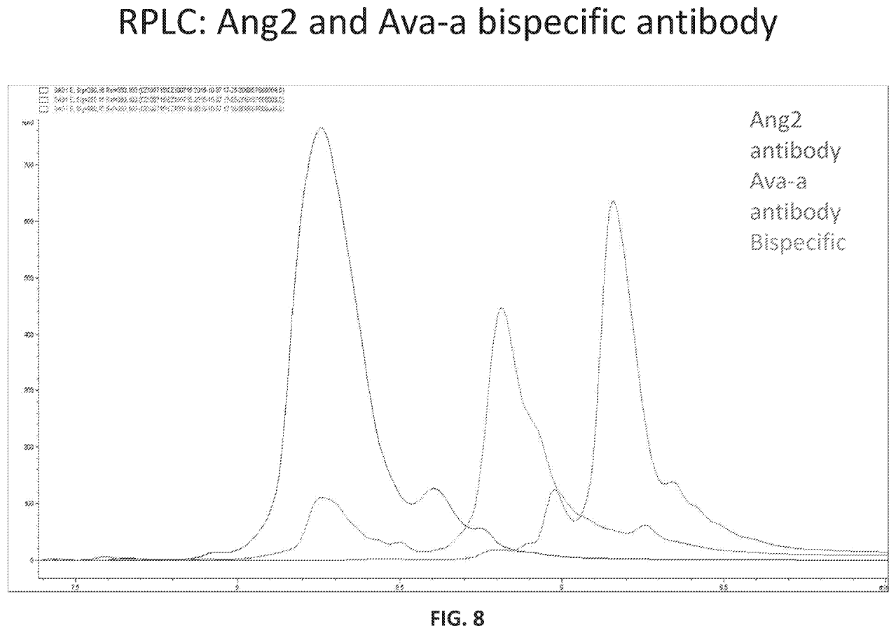

FIG. 8 provides the RPLC of a mixture of the bispecific antibody of Table 14 (1,3-dichloroacetone as the linker reagent of General procedure B, X-L-X) with Bevacizumab antibody and Anti-Ang2 antibody demonstrating that the bispecific antibody of Table 14 is not Bevacizumab or Anti-Ang2.

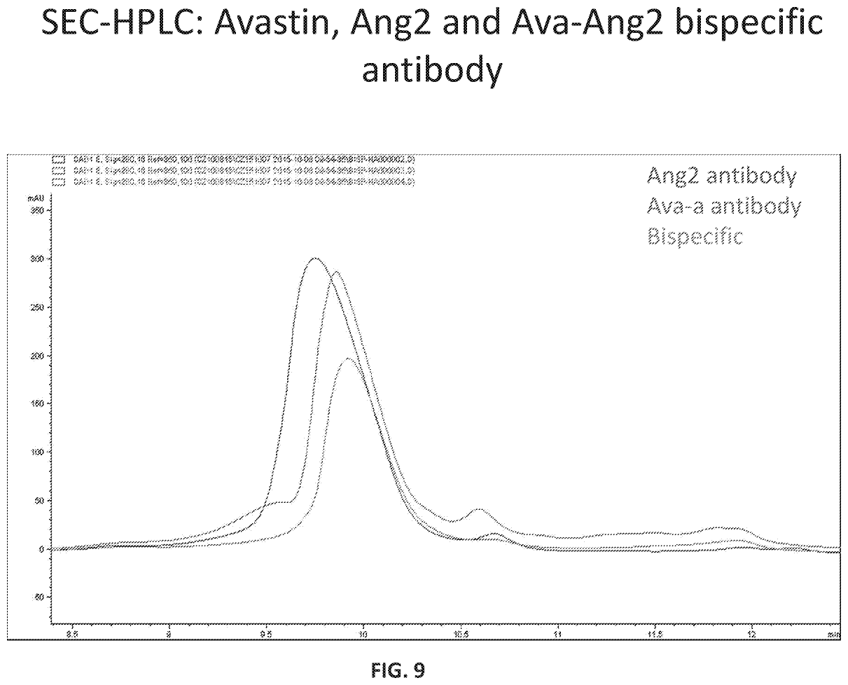

FIG. 9 provides the SEC-HPLC of a mixture of the bispecific antibody of Table 14 with Bevacizumab antibody and Anti-Ang2 antibody demonstrating that the bispecific antibody of Table 14 is not Bevacizumab antibody or Anti-Ang2 antibody

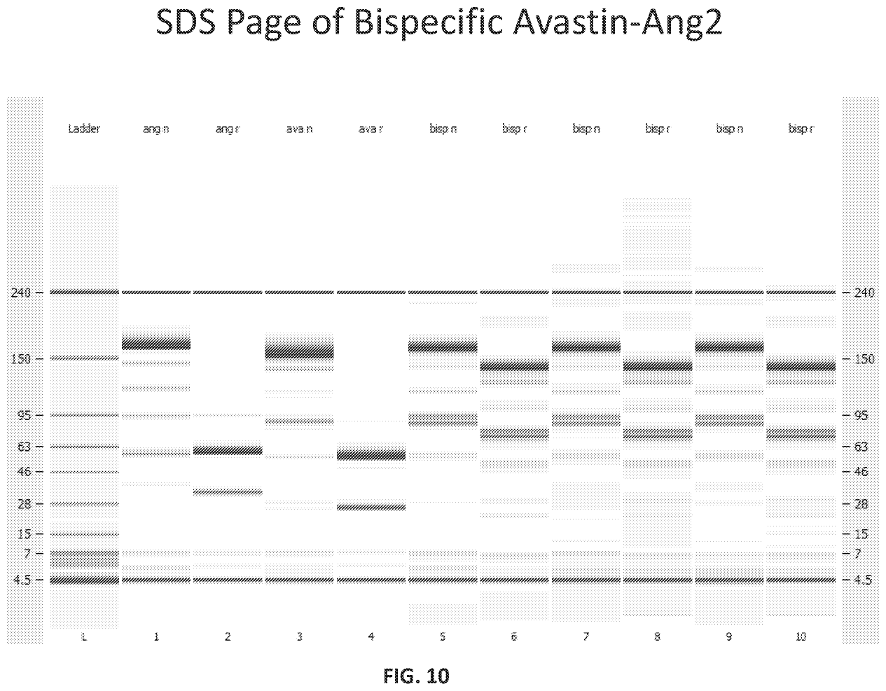

FIG. 10 provides SDS Page comparing bispecific antibody of Table 14 with Bevacizumab antibody and Anti-Ang2 antibody, each in reduced and non-reduced form.

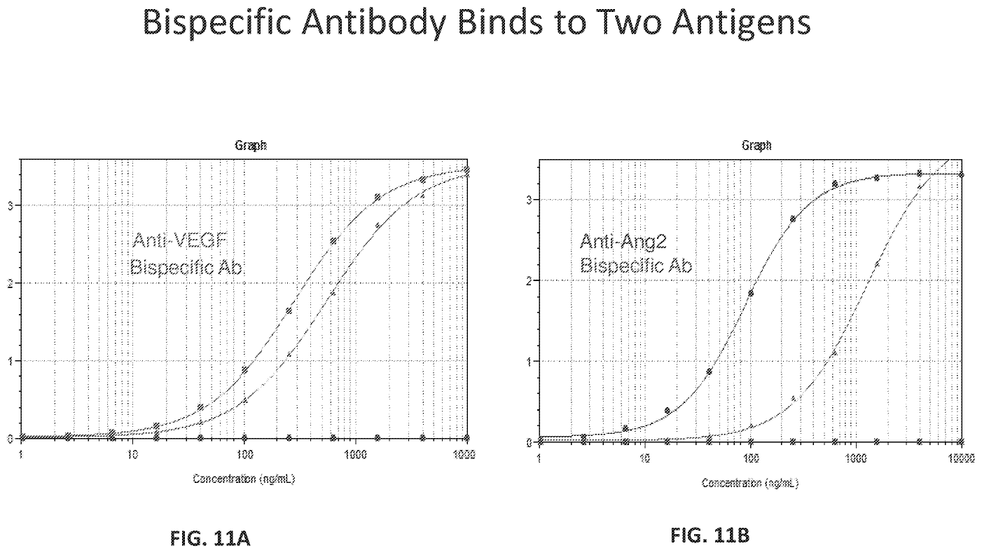

FIG. 11A-B provides a graph demonstrating the bispecific antibody of Table 14 binds to VEGF antigen and a graph demonstrating the bispecific antibody of Table 14 binds to Anti-Ang2 antigen whereas Bevacizumab only binds to the Anti-VEGF antigen and Anti-Ang2 only binds to the Anti-Ang2 antigen.

As shown in FIG. 11A, binding to VEGF was tested by adding between 1 ng/mL and 1000 ng/mL of VEGF and Ang2. Anti-VEGF and bispecific antibody were both shown to be effective binders to VEGF. Anti-VEGF and the bispecific Antibody exhibited similar shaped binding curves across the titration. The binding effect of Anti-VEGF to the bispecific Antibody leveled off around 1000 ng/mL, with Anti-VEGF exhibiting similar binding at that concentration. No binding of Anti-Ang2 to VEGF was detected.

As shown in FIG. 11B, binding to Ang2 was tested by adding between 1 ng/mL and 10,000 ng/mL of VEGF and Ang2. Anti-Ang2 and bispecific Antibody were shown to be effective binders to Ang2. No binding of Anti-VEGF to Ang2 was detected.

### Example 7

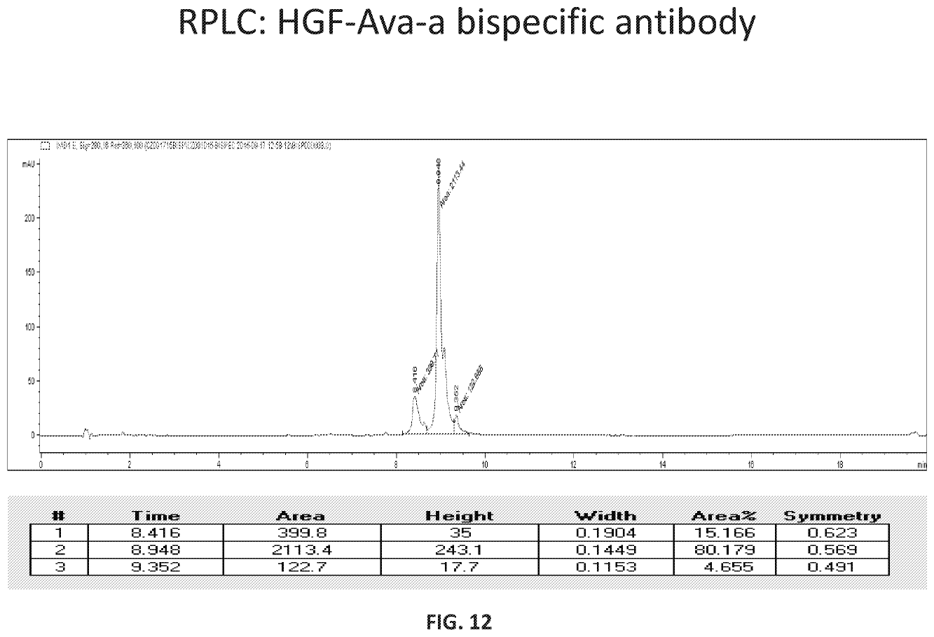

FIG. 12 provides the RPLC of the bispecific antibody of Table 15.

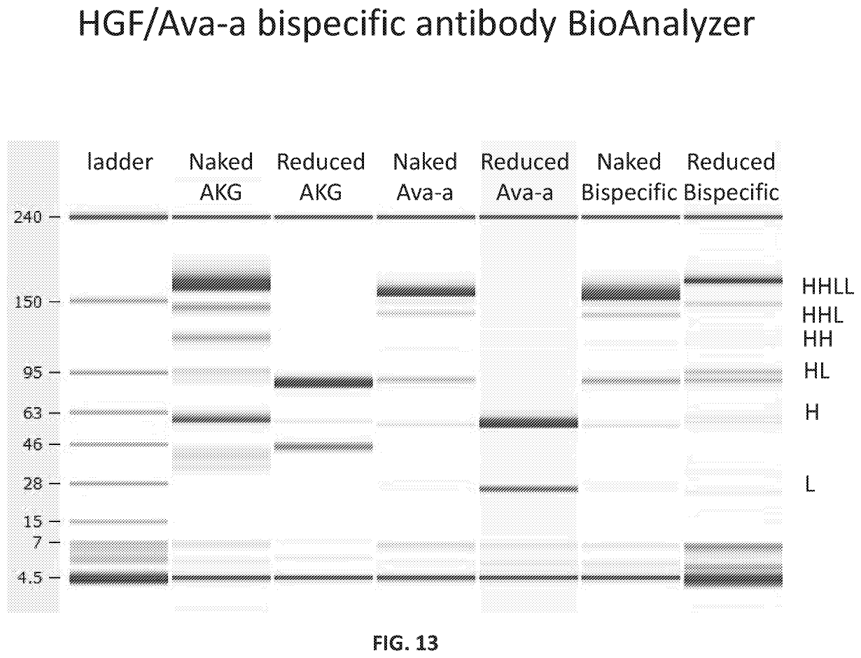

FIG. 13 provides Bio-Analyzer Analysis comparing bispecific antibody of Table 15 with Anti-HGF and Bevacizumab, each in reduced and non-reduced form.

### Example 8

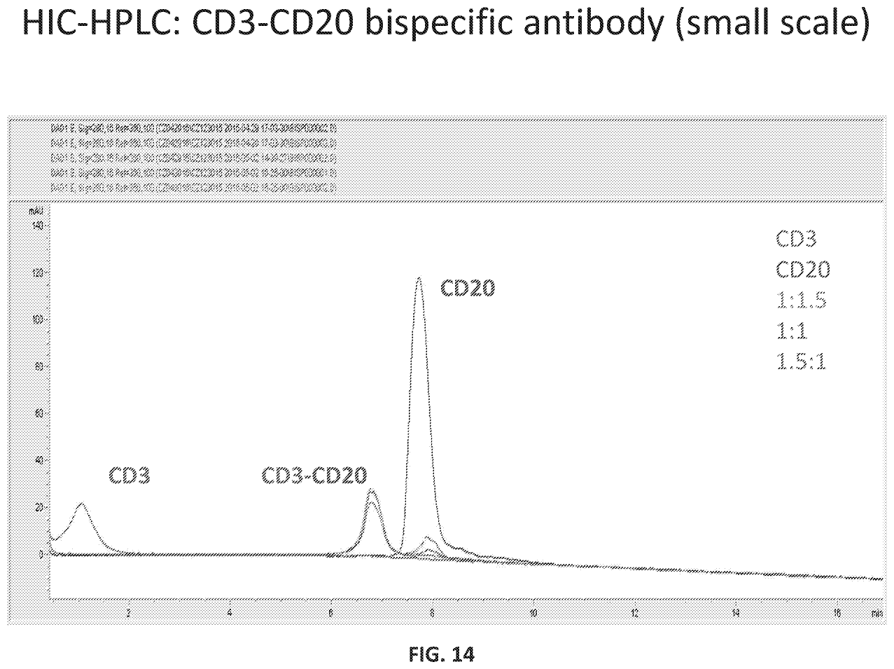

FIG. 14 provides the HIC-HPLC of the bispecific antibody of Table 16 (4,5-di(bromomethyl)-2-methyl-triazole or 1,3-dichloroacetone as the linker reagent of General procedure B, X-L-X) with Anti-CD3 and Anti-CD20 demonstrating that the bispecific antibody of Table 16 is not Anti-CD3 or Anti-CD20.

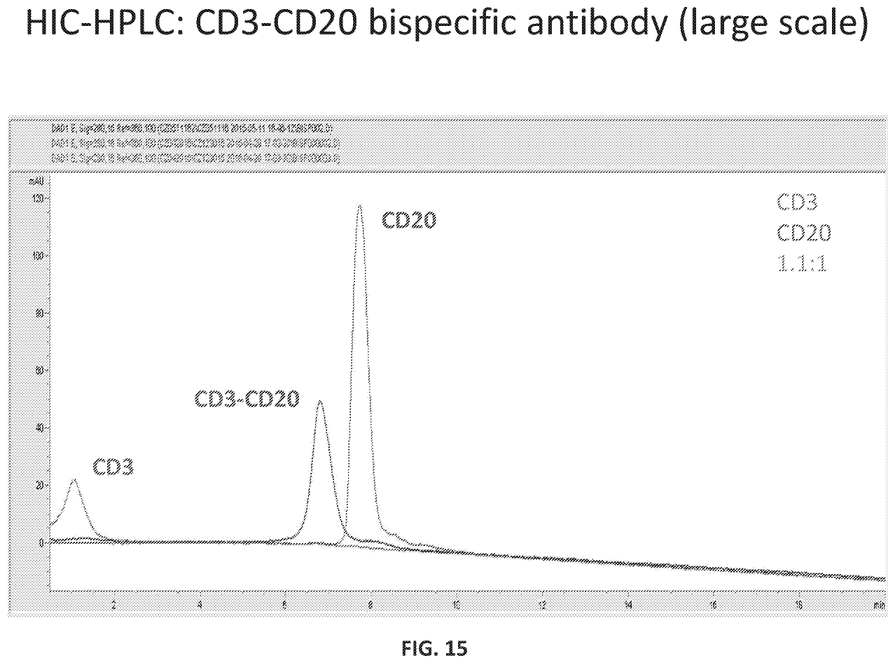

FIG. 15 provides the HIC-HPLC of a mixture of the bispecific antibody of Table 16 (4,5-di(bromomethyl)-2-methyl-triazole or 1,3-dichloroacetone as the linker reagent of General procedure B, X-L-X) with a mixture of Anti-CD3 antibody and Anti-CD20 antibody demonstrating that the bispecific antibody of Table 16 is not Anti-CD3 antibody or Anti-CD20 antibody. The ratio of the mixture of LCA-SH and HCA-SH from Anti-CD3 and the mixture of LCB-SH and HCB-SH from Anti-CD20 that were reacted together according to General procedure B is 1.1:1.

### Example 9

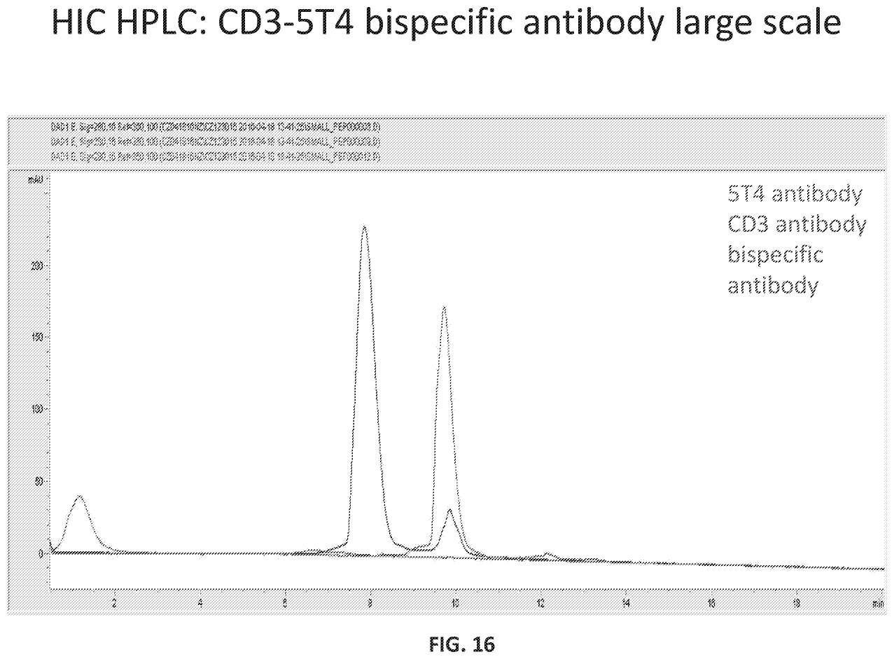

FIG. 16 provides the HIC-HPLC of the bispecific antibody of Table 17 (2,6-dibromomethyl-pyridine or 1,3-dichloroacetone as the linker reagent of General procedure B, X-L-X) with Anti-CD3 antibody and Anti-5T4 antibody demonstrating that the bispecific antibody of Table 17 is not Anti-CD3 antibody or Anti-5T4 antibody.

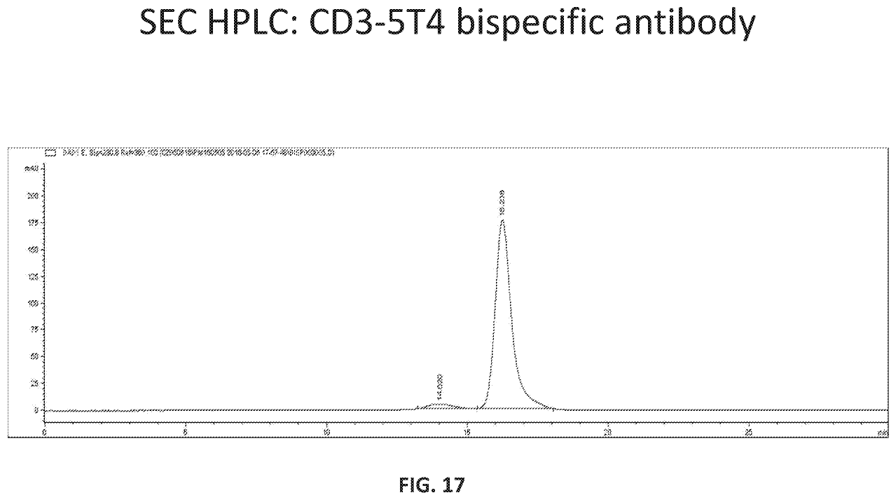

FIG. 17 provides the SEC HPLC of the bispecific antibody of Table 17 synthesized according to General procedure B using (2,6-dibromomethyl-pyridine or 1,3-dichloroacetone as X-L-X.

### Example 10

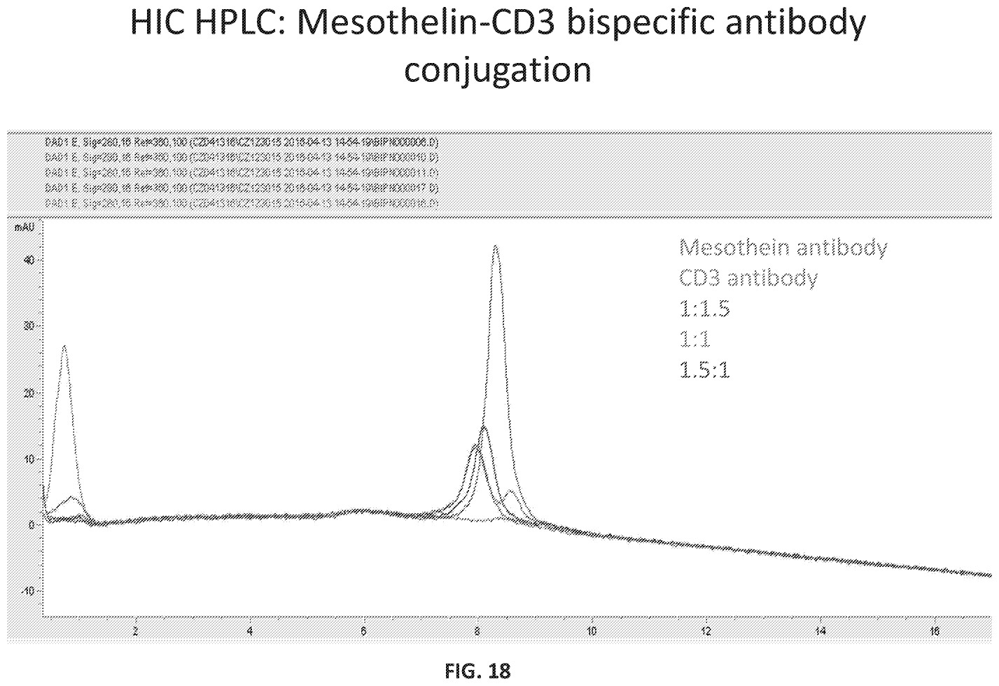

FIG. 18 provides the HIC-HPLC of the bispecific antibody of Table 18 (4,5-di(bromomethyl)-2-methyl-triazole or 1,3-dichloroacetone as the linker reagent of General procedure B, X-L-X). The mixture of LCA-SH and HCA-SH from Anti-mesothelin and the mixture of LCB-SH and HCB-SH from Anti-CD3 were reacted together in three different ratios, 1.5:1, 1:1 and 1:1.5.

### Example 11

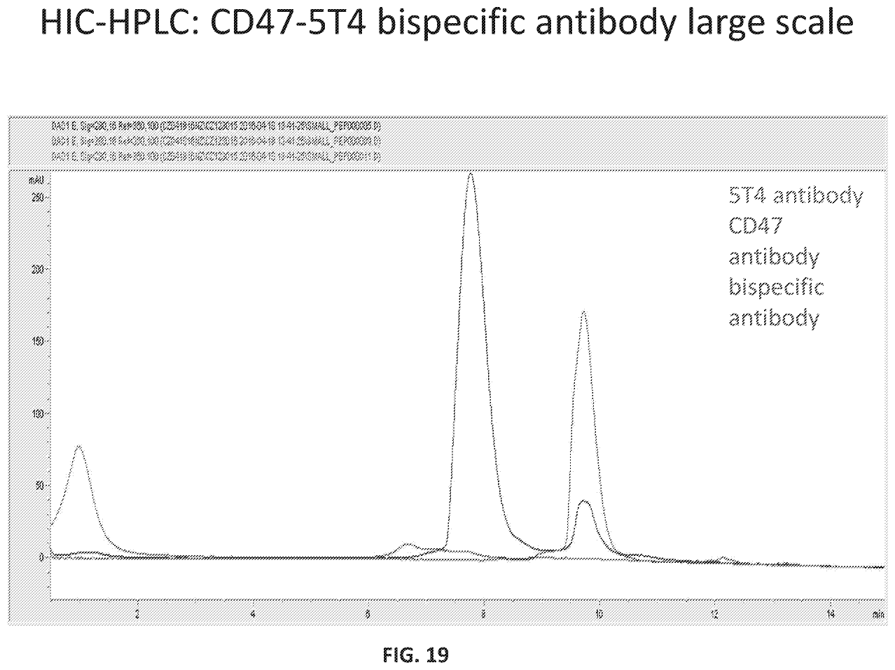

FIG. 19 provides the HIC-HPLC of the bispecific antibody of Table 19 (1,3-dichloroacetone as the linker reagent of General procedure B, X-L-X) with Anti-CD47 antibody and Anti-5T4 antibody demonstrating that the bispecific antibody of Table 19 is not Anti-CD47 antibody or Anti-5T4 antibody.

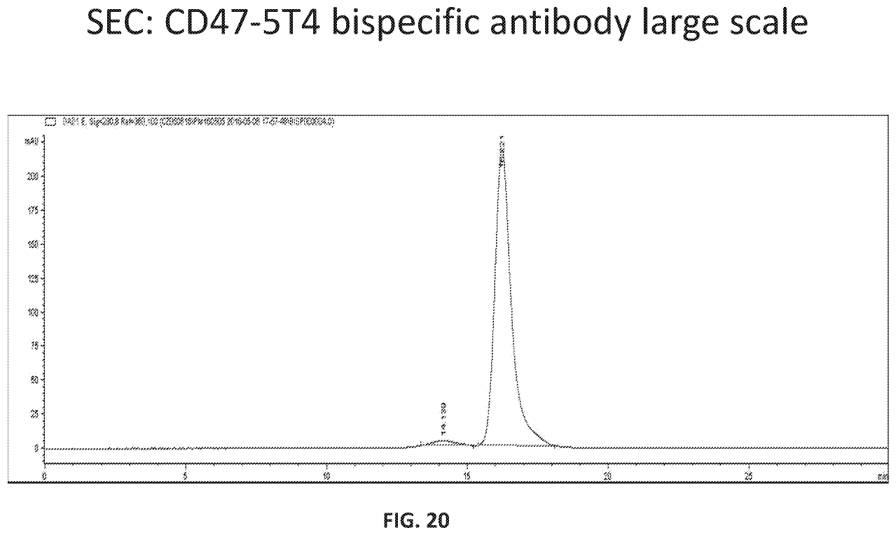

FIG. 20 provides the SEC HPLC of the bispecific antibody of Table 19 (1,3-dichloroacetone as the linker reagent of General procedure B, X-L-X) synthesized according to General procedure B.

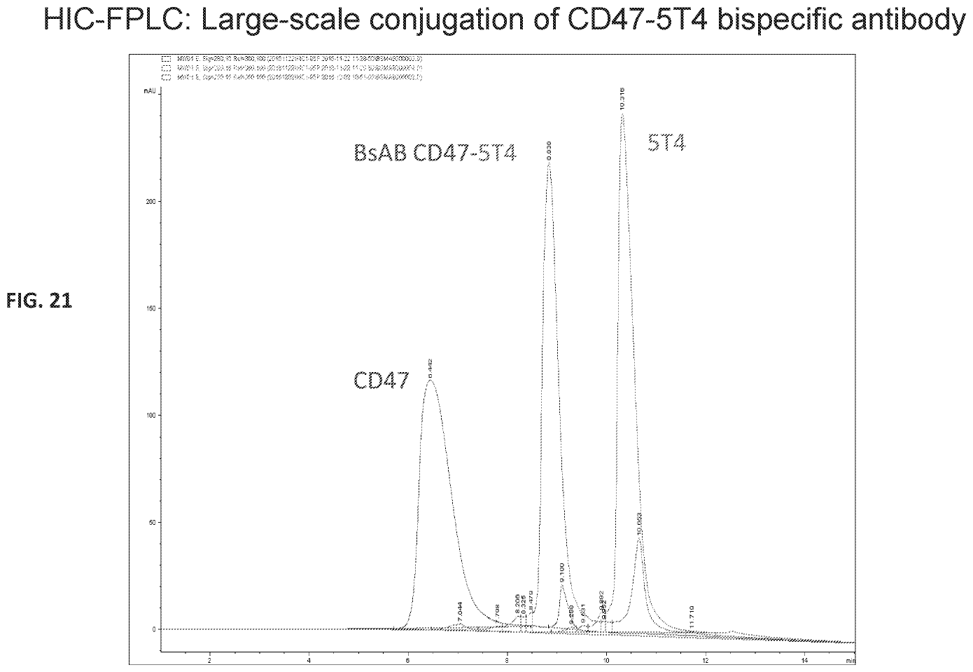

FIG. 21 provides the HIC-FPLC of a large scale synthesis of the bispecific antibody of Table 19 (1,3-dichloroacetone as the linker reagent of General procedure B, X-L-X) with Anti-CD47 antibody and Anti-5T4 antibody demonstrating that the bispecific antibody of Table 19 is not Anti-CD47 antibody or Anti-5T4 antibody.

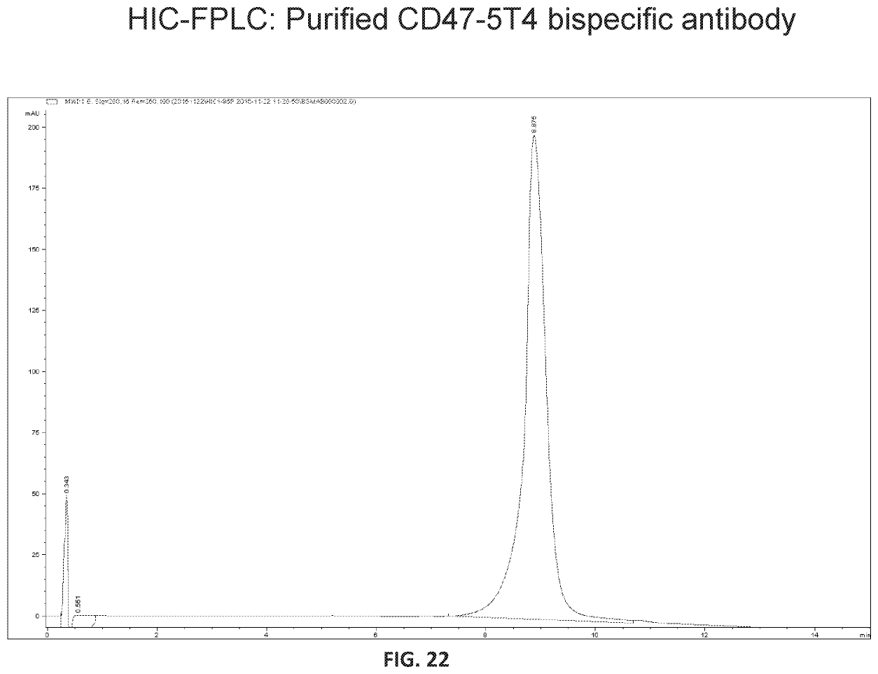

FIG. 22 provides the HIC-FPLC of a large scale synthesis of the bispecific antibody of Table 19 synthesized according to General procedure B using 1,3-dichloroacetone as X-L-X.

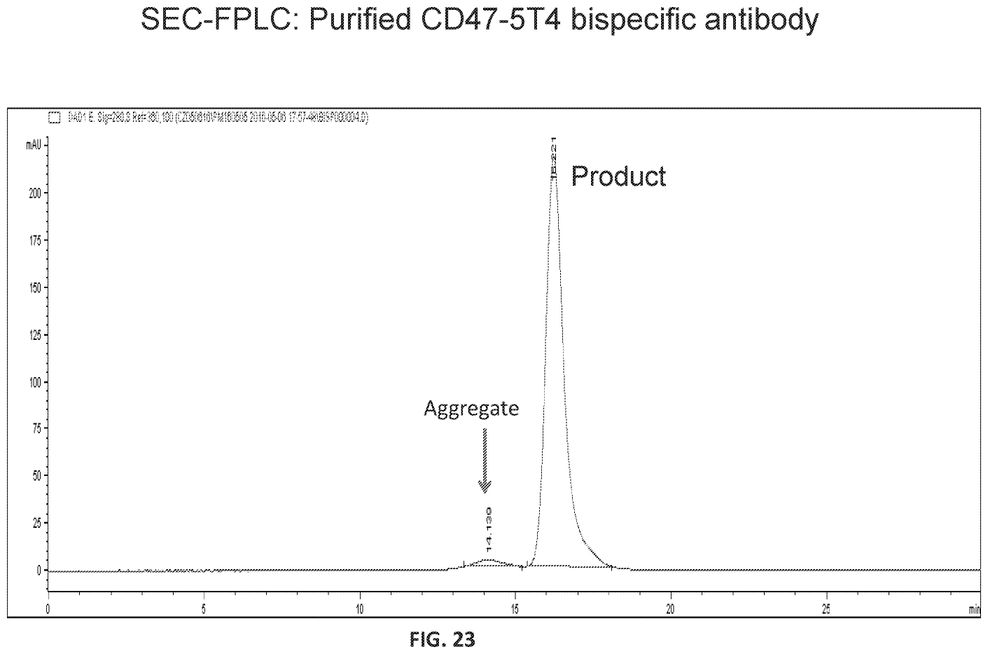

FIG. 23 provides the SEC-FPLC of a large scale synthesis of the bispecific antibody of Table 19 synthesized according to General procedure B using 1,3-dichloroacetone as X-L-X.

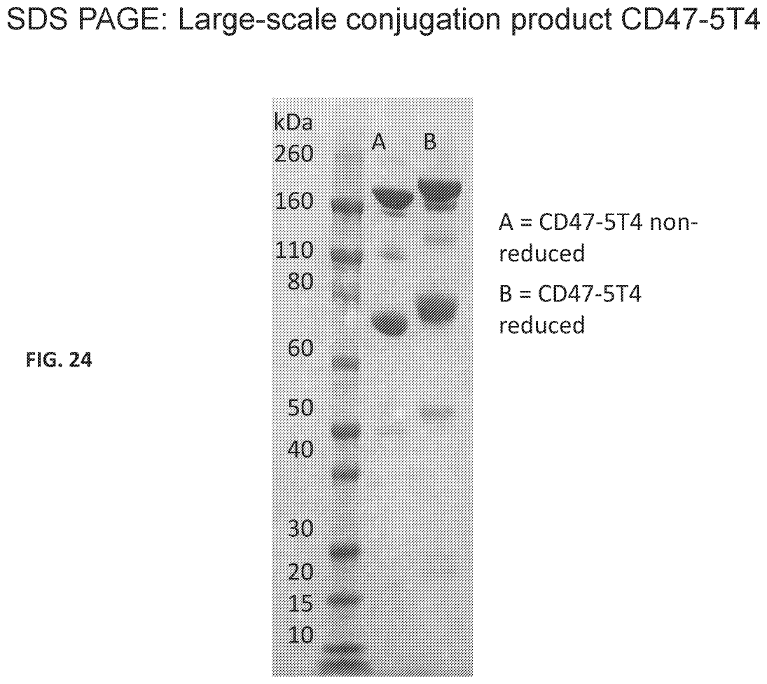

FIG. 24 provides the SDS page of a large scale synthesis of the bispecific antibody of Table 19 (1,3-dichloroacetone as the linker reagent of General procedure B, X-L-X) in reduced and non-reduced form.

### Example 12

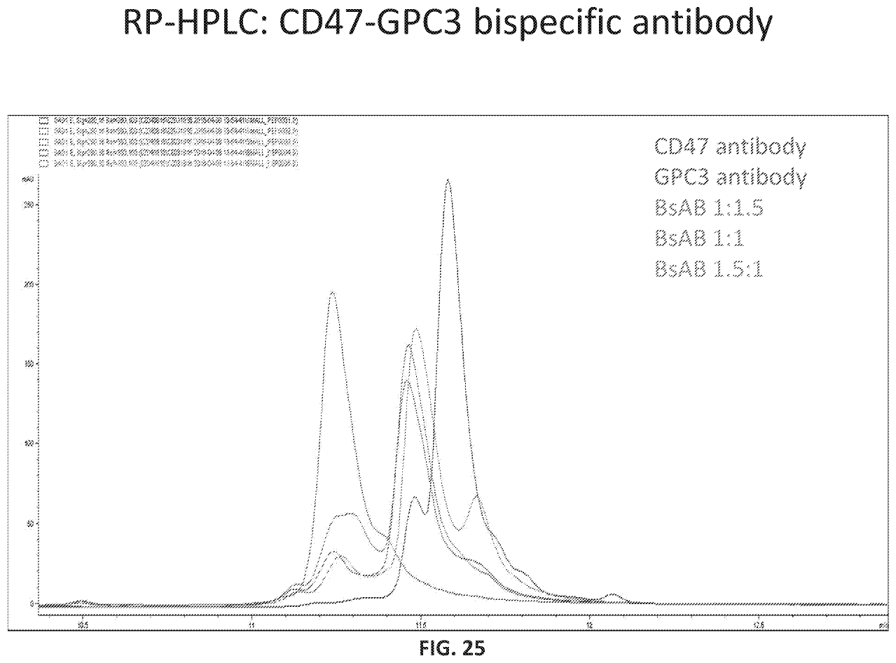

FIG. 25 provides the RP-HPLC of the bispecific antibody of Table 20 (2,4-dibromo-3-pentanone or 1,3-dichloroacetone as the linker reagent of General procedure B, X-L-X) with Anti-CD47 antibody and Anti-GPC3 antibody demonstrating that the bispecific antibody of Table 20 is not Anti-CD47 antibody or Anti-GPC3 antibody.

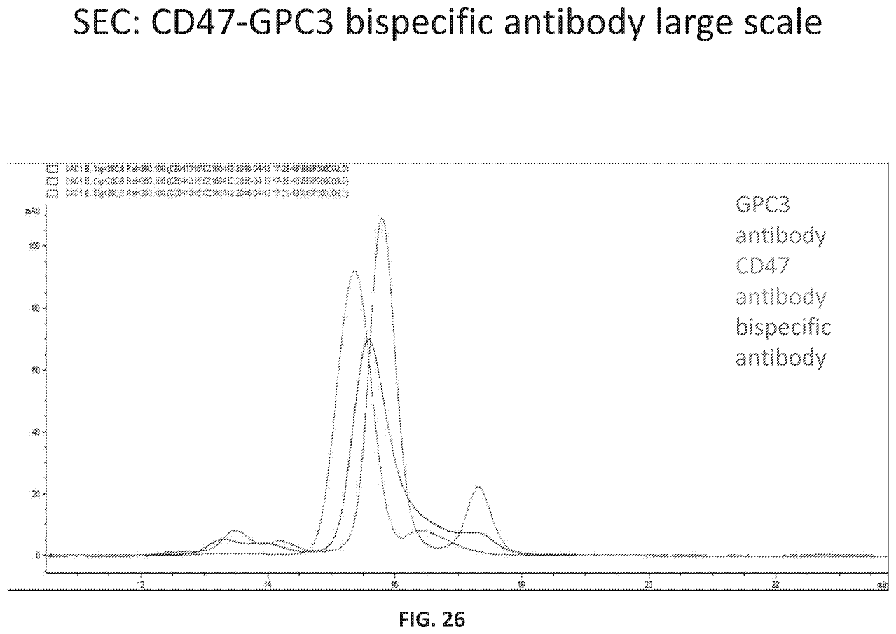

FIG. 26 provides the SEC HPLC of the bispecific antibody of Table 20 (2,4-dibromo-3-pentanone or 1,3-dichloroacetone as the linker reagent of General procedure B, X-L-X) with Anti-CD47 antibody and Anti-GPC3 antibody demonstrating that the bispecific antibody of Table 20 is not Anti-CD47 or Anti-GPC3.

### Example 13

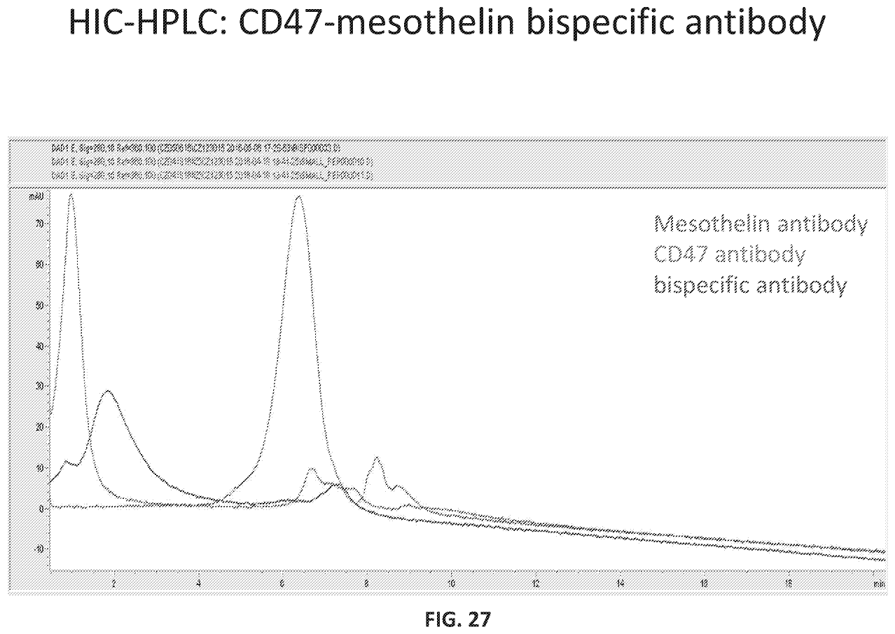

FIG. 27 provides the HIC-HPLC of the bispecific antibody of Table 21 (2,6-dibromomethyl-pyridine or 2,4-dibromo-3-pentanone as the linker reagent of General procedure B, X-L-X) with Anti-CD47 antibody and Anti-mesothelin antibody demonstrating that the bispecific antibody of Table 21 is not Anti-CD47 or Anti-mesothelin.

### Example 14

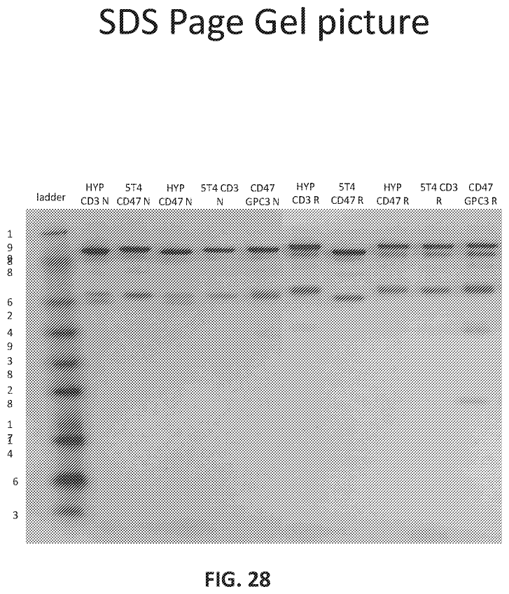

FIG. 28 provides a SDS page gel.

### Example 15

### Bispecific Antibody Binds to Two Antigens

An antibody titration was performed in order to compare the ability to bind different concentrations of VEGF and Ang2 (angiopoietin 2) of Anti-VEGF antibody, Anti-Ang2 antibody, and a bispecific antibody for VEGF and Ang2. As shown in FIG. 11A, binding to VEGF was tested by adding between 1 ng/mL and 1000 ng/mL of VEGF and Ang2. Anti-VEGF and bispecific antibody were both shown to be effective binders to VEGF. Anti-VEGF and the bispecific Antibody exhibited similar shaped binding curves across the titration. The binding effect of Anti-VEGF to the bispecific Antibody leveled off around 1000 ng/mL, with Anti-VEGF exhibiting similar binding at that concentration. No binding of Anti-Ang2 to VEGF was detected.

As shown in FIG. 11B, binding to Ang2 was tested by adding between 1 ng/mL and 10,000 ng/mL of VEGF and Ang2. Anti-Ang2 and bispecific Antibody were shown to be effective binders to Ang2. No binding of Anti-VEGF to Ang2 was detected.

### Example 16

### Cytotoxicity Assay Results of Bispecific CD3-Mesothelin

The cytotoxicity of antibodies that target only mesothelin and bispecific antibodies that target CD3 and Mesothelin was measured by determining cell viability of peripheral mononuclear blood cells (PMBCs) and NCI-H226 cells (derived from the lung).

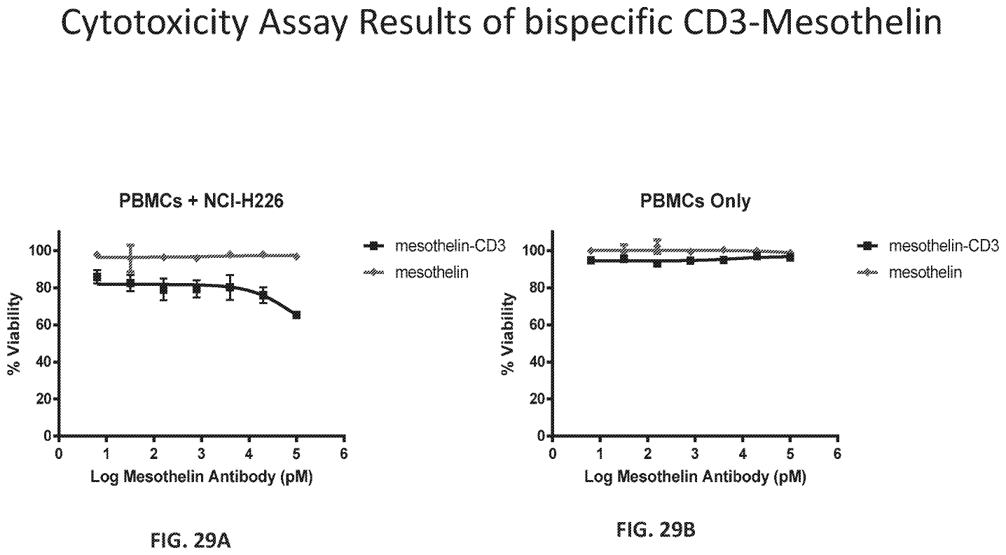

In FIG. 29A, antibodies specific to mesothelin were tested along with bispecific antibodies targeting both mesothelin and CD3. The cells types tested for viability were PMBCs and NCI-H226s (E:T=5:1). The titration of both types of antibody showed high viability at the levels tested from about log=1 pM antibody to about log=5 pM antibody. Antibody targeting mesothelin alone exhibited high (near 100%) viability at all antibody concentrations tested. Cell viability with the bispecific antibody was slightly lower and decreased at the higher antibody concentrations tested.

In FIG. 29B, antibodies specific to mesothelin were tested along with bispecific antibodies targeting both mesothelin and CD3. The cells types tested for viability were PMBCs. The titration of both types of antibody showed high viability at the levels tested from about log(1) pM antibody to about log(5) pM antibody. Antibody targeting mesothelin alone showed cell viability of near 100%. The cell viability as a result of testing with the bispecific antibody was slightly lower at most concentrations tested.

### Example 17

### The Effect of Effector to Target Cell Ratios on Cytotoxicities of Bispecific Antibody CD47-5T4

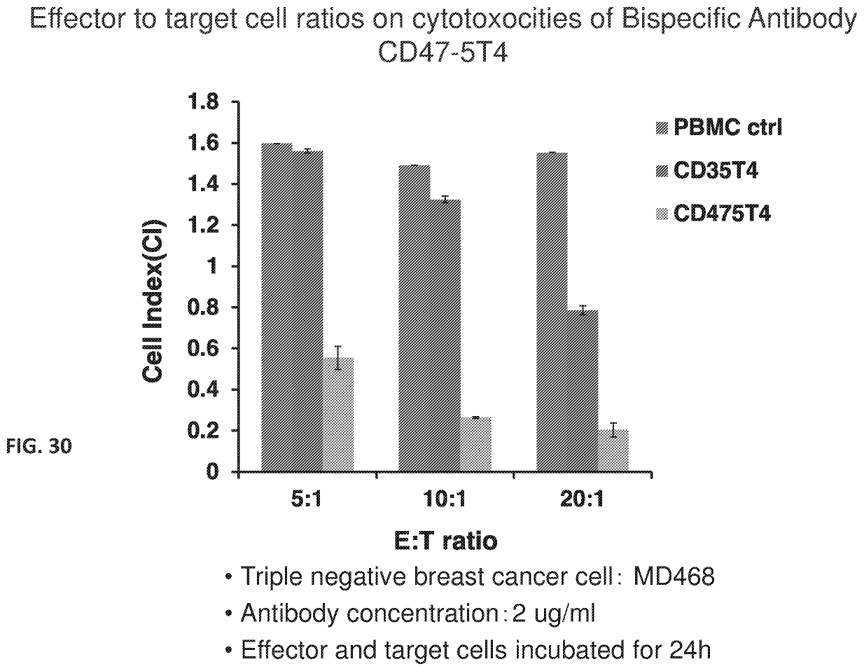

The cytotoxicites of bispecific antibodies CD47-5T4 and CD35-T4 were measured in MD468 cells (Triple negative breast cancer cell). The results are shown in FIG. 30. The antibody concentrations used were 2 μg/ml and the antibodies were incubated with the cells for 24 hours. Cell cytotoxicities were measured at three ratios of effector to target cells, 5:1, 10:1, and 20:1. Cytotoxicities were measured using a cell index. A PBMC control cell line was used that showed little variation in cell viability across the effector to target cell ratios used. Higher cytotoxicity (as measured by a decreasing cell index) was seen for the CD47-5T4 and CD35-T4 antibody as the ratio of effector to target cells increased. The greatest cytotoxicity seen was for CD47-5T4 antibody at an effector to target ration of 20:1.

### Example 18

### Cytotoxicities of Bispecific Antibodies CD47-5T4 and CD3-5T4

The cytotoxicities of bispecific antibodies CD47-5T4 and CD3-5T4 were tested in various cancer cell type models and compared to antibodies with only one target.

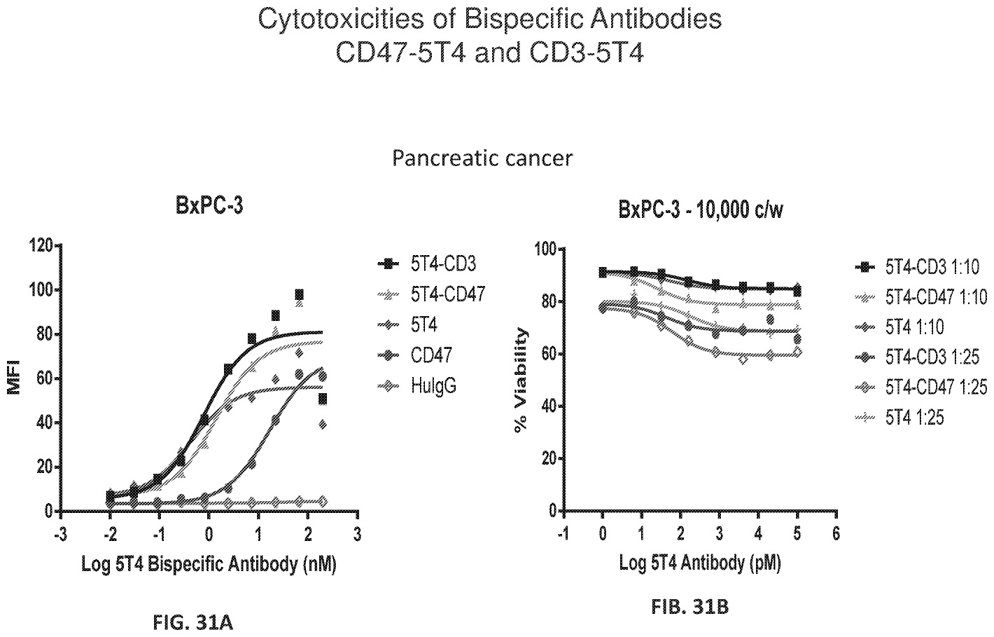

In FIG. 31A, bispecific antibodies 5T4-CD3 and 5T4-CD47 were tested to determine percent cell viability and mean fluorescence intensity (MFI) along with single target antibody controls 5T4, CD47, and humanIgG (HuIgG) The cells tested were BxPC-3 (pancreas) cells. The MFI did not change with an increase in HuIgG antibody. The MFI was highest for the 5T4-CD3 and 5T4-CD47 antibodies at high antibody concentration, tested between about −2 and 2 log antibody (nM). The MFI was slightly lower for CD47 and 5T4 antibodies at high concentrations of 5T4 Bispecific Antibody

In FIG. 31B, various effector target cells ratios were tested using 5T4-CD3, 5T4-CD47, and 5T4 antibodies to determine cell viability in BxPC-3 cells at 10,000 c/w. The effector (PBMCs) to target cell (BxPC-3) ratios used were 1:10 and 1:25. Cell viability was measured over a titration of between log=0 and log=5 antibody (pM). Across all antibody types, the 1:10 effector to target ratios resulted in higher cell viability. At both target to effector concentrations, cell viability as a result of bispecific antibody treatment was only slightly lower than single target 5T4 antibody.

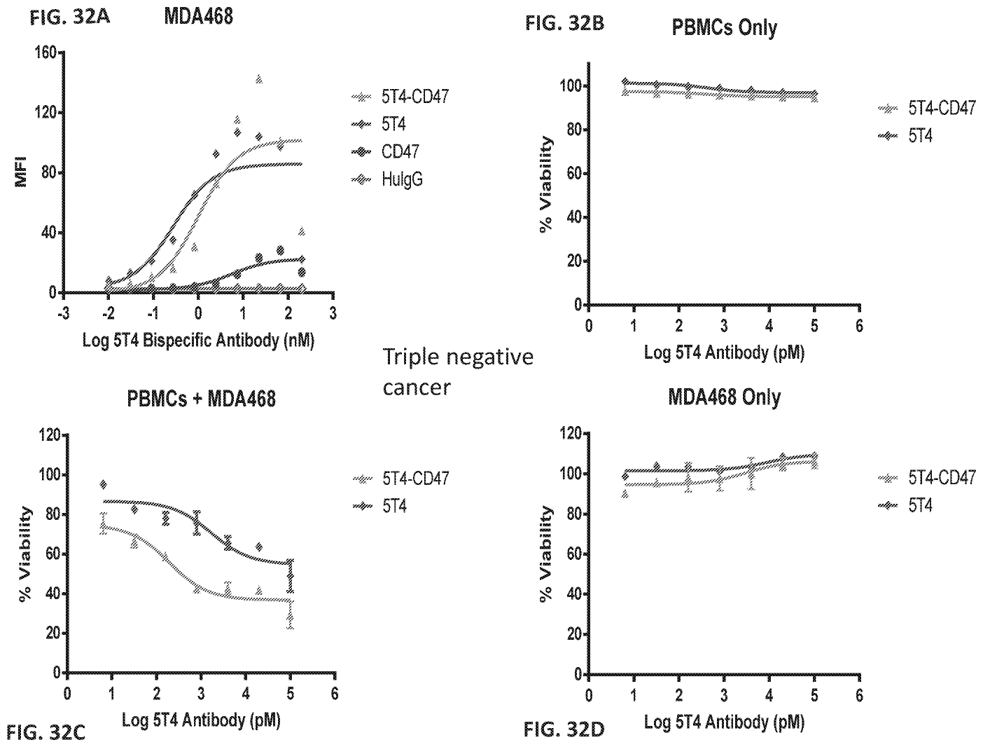

In FIG. 32A-D, the effect of 5T4-CD47 bispecific antibody versus 5T4 and CD47 antibody is measured in MDA468 (triple negative breast cancer) cells and PBMCs. MFI was measured for 5T4-CD47, 5T4, CD47, and HuIgG antibodies across about −2 to 3 log antibody (nM) for MDA468 cells. At high concentration, the greatest MFI increase was seen for 5T4-CD47 antibody treatment, slightly higher than 5T4 and much higher than CD47. The MFI did not change with an increase in HuIgG antibody. Next, cell viability was determined in PMBC cells using either 5T4-CD47 antibody or 5T4 antibody with similar viability seen for antibody concentrations between log 1 and log 5 pM. In testing PBMCs (effector)+MDA468 (target), cell viability with treatment of 5T4-CD47 antibody was lower than with 5T4 antibody. In testing 5T4-CD47 and 5T4 antibodies with MDA468 cells only, the decrease in cell viability with 5T4-CD47 antibody was only slight as compared to 5T4 antibody.

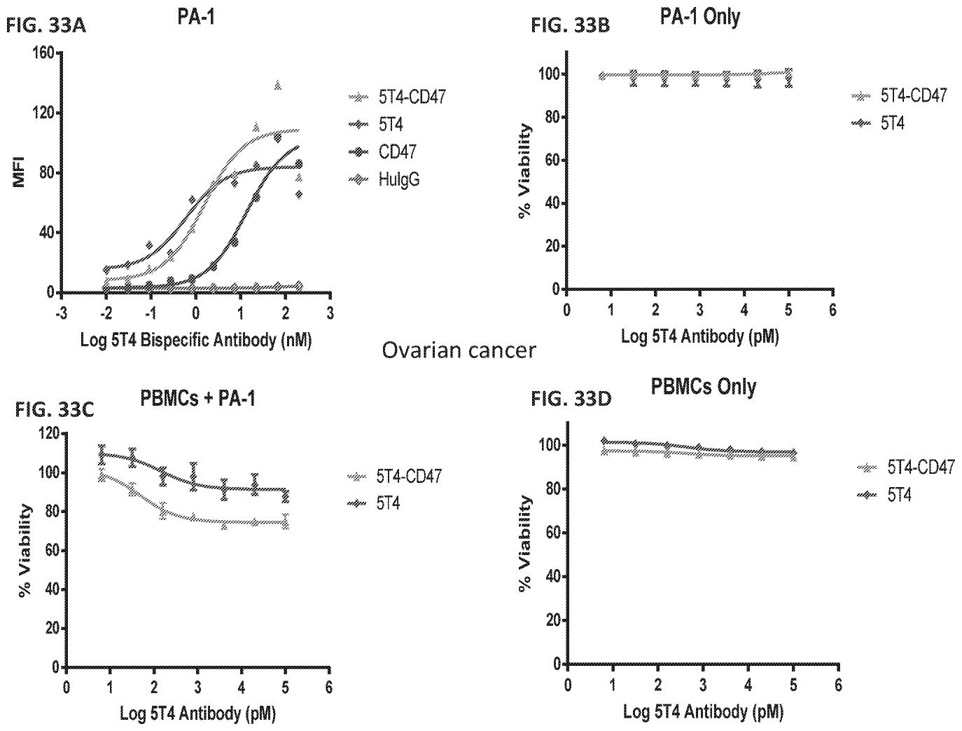

In FIG. 33A-D, the effect of 5T4-CD47 bispecific antibody versus 5T4 and CD47 antibody is measured in PA-1 (ovarian cancer) cells and PBMCs. MFI was measured for 5T4-CD47, 5T4, CD47, and HuIgG antibodies across about −2 to 3 log antibody (nM) for PA-1 cells. At high antibody concentration, the greatest MFI increase was seen for 5T4-CD47 antibody treatment, slightly higher than CD47 and 5T4. The MFI did not change with an increase in HuIgG antibody. Next, cell viability was determined in PA-1 cells using either 5T4-CD47 antibody or 5T4 antibody with similar viability seen for antibody concentrations between log 1 and log 5 pM. In testing PBMCs (effector)+PA-1 (target) cell viability with treatment of 5T4-CD47 antibody was lower than with 5T4 antibody. In testing 5T4-CD47 and 5T4 antibodies with PBMC cells only, the decrease in cell viability with 5T4-CD47 antibody was only slight as compared to 5T4 antibody treatment.

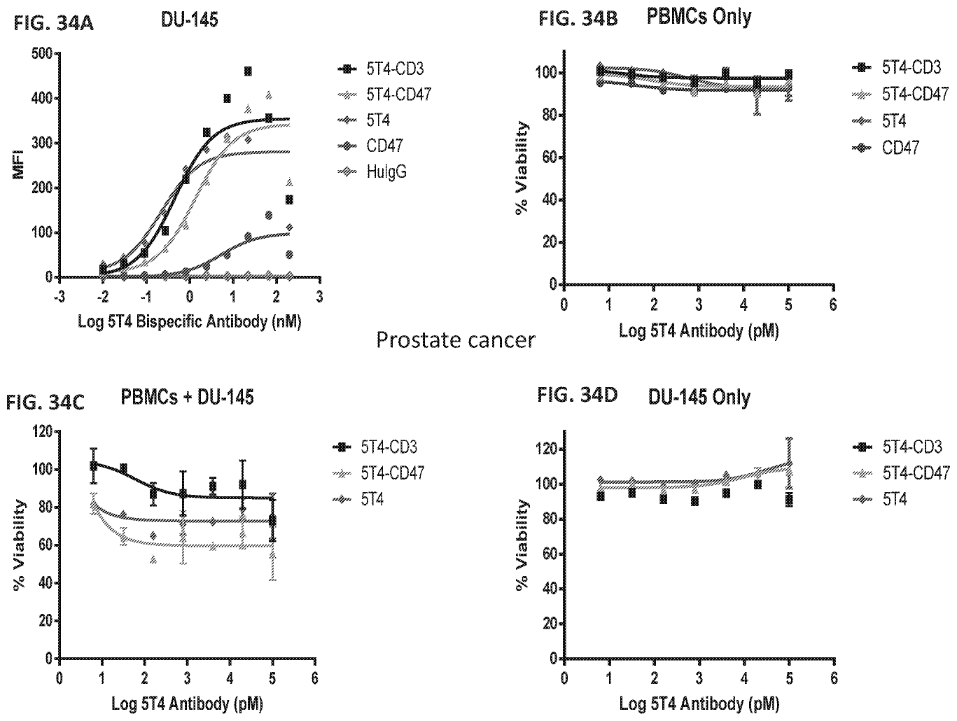

In FIG. 34A-D, the effect of 5T4-CD47 and 5T4-CD3 bispecific antibodies versus 5T4 and CD47 antibody is measured in DU-145 (prostate cancer) cells and PBMCs. MFI was measured for 5T4-CD47, 5T4-CD3, 5T4, CD47, and HuIgG antibodies across about −2 to 3 log antibody (nM) for DU-145 cells. At high antibody concentration, the greatest MFI increase was seen for 5T4-CD3, followed by 5T4-CD47. These values were slightly higher than 5T4 antibody and much higher than CD47 antibody. The MFI did not change with an increase in HuIgG antibody. Next, cell viability was determined in PBMC cells using 5T4-CD47, 5T4-CD3, 5T4, or CD47 antibodies with similar viability seen across all antibody types for antibody concentrations between log 1 and log 5 pM. In testing PBMCs (effector)+DU-145 (target), cell viability was highest with 5T4-CD3 antibody, then 5T4 antibody, and lowest with 5T4-CD47 antibody. For cell viability testing in DU-145 cells, high viability (near 90 or 100 percent) was seen for 5T4-CD3, 5T4-CD47, and 5T4 antibodies across all concentrations tested.

### Example 19

### CD47-5T4 Antibody Selectively Killed Triple Negative Breast Cancer Cell MDA 231

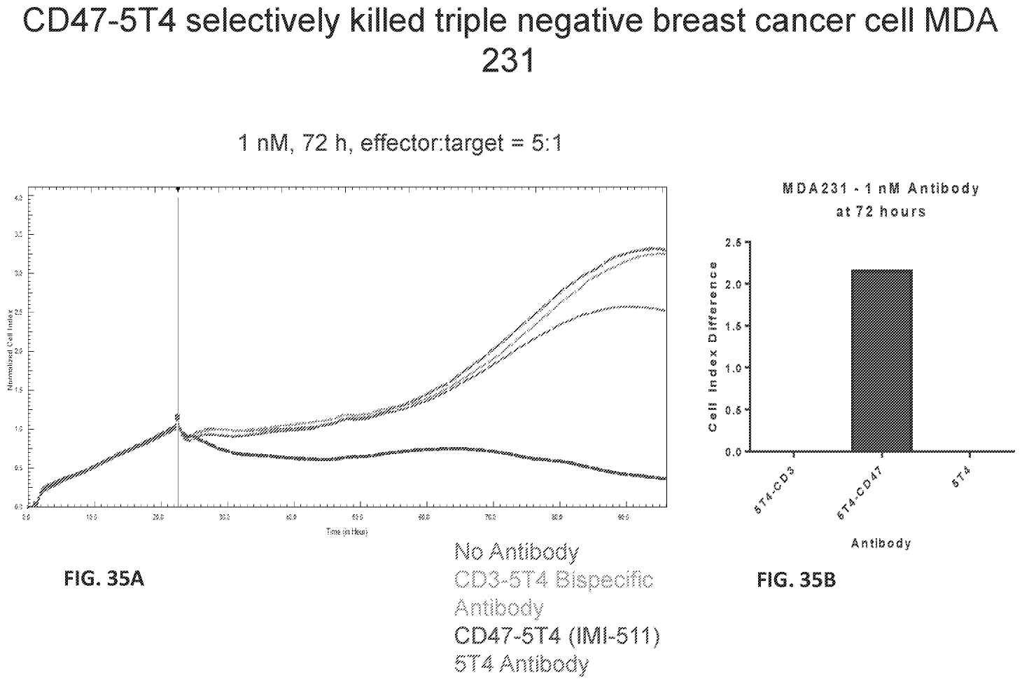

FIG. 35A-B. The effect of treating MDA 231 triple negative breast cancer cells with 1 nM of various antibodies was tested over 72 hours. In all instances the ratio of effector cells to target cells was 5:1. Testing included no antibody, CD3-5T4 bispecific antibody, CD47-5T4 bispecific antibody, and 5T4 antibody. At 72 hours, the cell viability, graphed as normalized cell index, was higher for 5T4 and CD3-5T4 bispecific antibody than for treatment with no antibody. Treatment with CD47-5T4 resulted in a marked decrease in cell viability as compared to all other treatment groups. The cell index difference was around 2 for treatment with CD47-5T4 at the seventy two hour mark.

### Example 20

### CD47-5T4 Selectively Killed Colon Cancer Cell LoVo

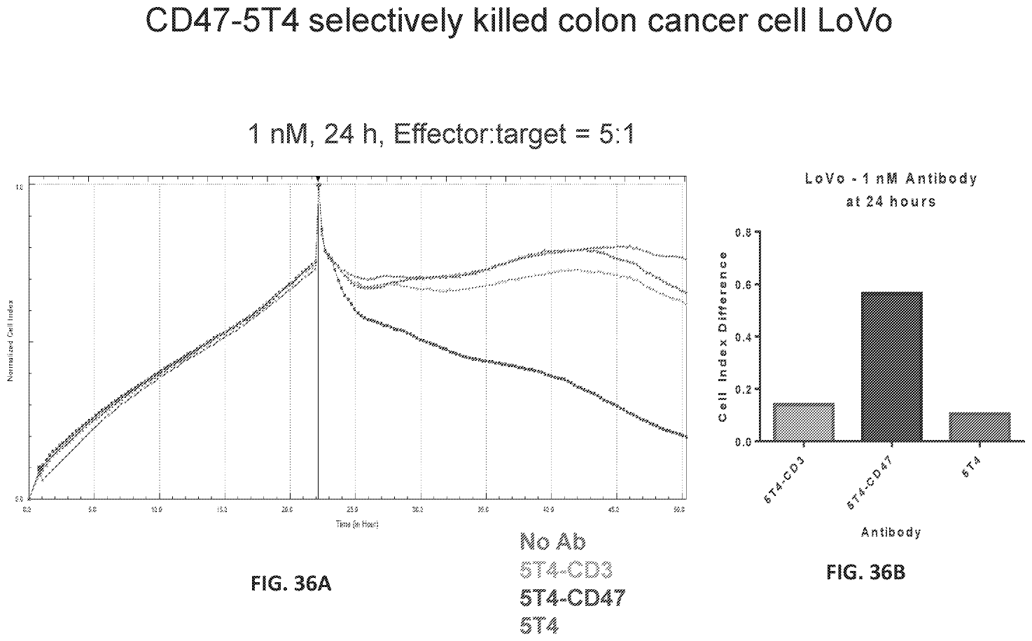

FIG. 36A-B. The effect of treating Lovo (colon cancer) cells with 1 nM of various antibodies was tested over 24 hours. In all instances the ratio of effector cells to target cells was 5:1. Testing included no antibody, 5T4-CD3, 5T4-CD47, and 5T4 antibody. At 24 hours, the cell viability, graphed as normalized cell index, was highest for treatment with no antibody, followed by 5T4 antibody, and 5T4-CD3 antibody. Treatment with 5T4-CD47 resulted in a marked decrease in cell viability as compared to all other treatment groups. The cell index difference for Lovo cells treated with 5T4-CD47 antibody at 24 hrs was around 0.6.

### Example 21

### CD47-5T4 Antibody Inhibited Tumor Growth in the Xenografted Lung Cancer H1975 Model

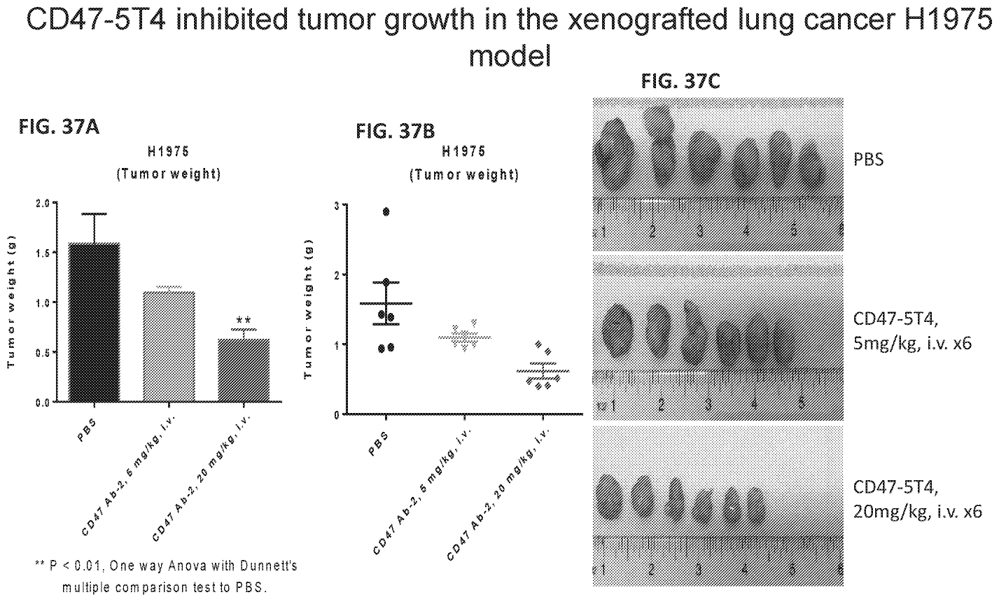

FIG. 37A-C. The effect of treatment with CD47-5T4 antibodies on tumor growth was tested in a mouse xenografted lung cancer H1975 model. Tumor weight for six tumor biopsies was measured for treatment with PBS, CD47-5T4 antibody at a concentration of 5 mg/kg, and CD47-5T4 antibody at a concentration of 20 mg/kg. A decrease in tumor weight was observed for both CD47-5T4 treatment groups as compared to treatment with PBS. The greatest decrease in tumor weight was observed for a treatment with 20 mg/kg of CD47-5T4 antibody.

### Example 22

### CD47-Trop2 Selectively Kill Triple Negative Breast Cancer Cell MDA 231

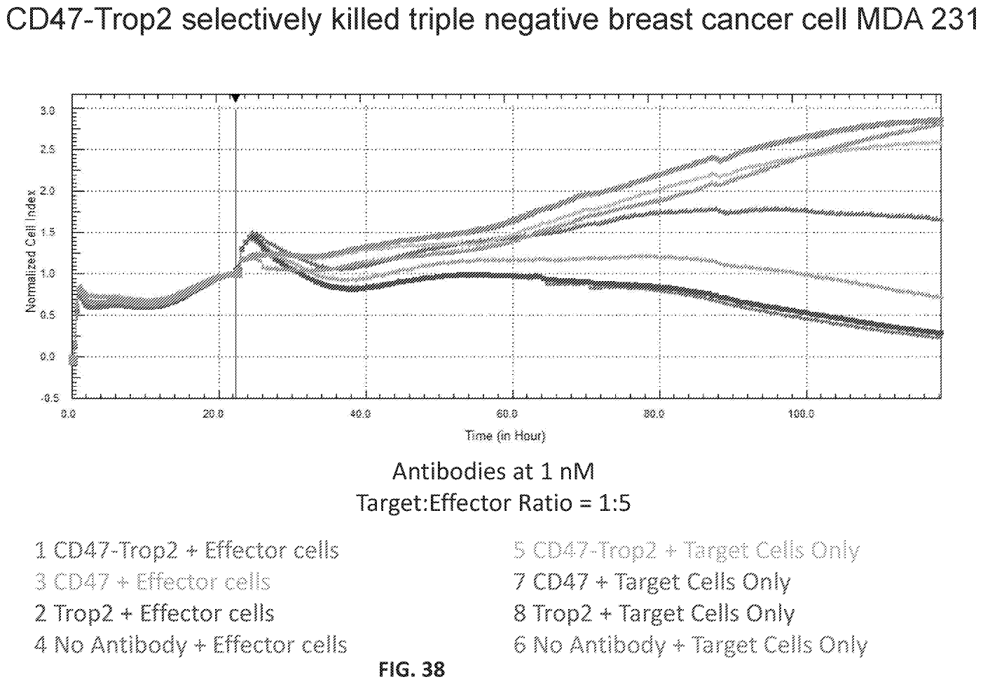

FIG. 38. The effect of treating MDA 231 (triple negative breast cancer) cells with 1 nM of various antibodies was tested over a time period of longer than 100 hours. The target:effector ratio used was 1:5. The antibodies types used were CD47-Trop2, CD47, Trop2, and no antibody. For each antibody type, testing was done with the addition of effector cells or the addition of target cells only. The antibody treatments with target cells only added served as controls and showed high normalized cell indexes. Both treatment with CD47-Trop2+Effector cells and Trop2+Effector cells showed a marked decrease in cell viability compared to controls, measured by the normalized cell index.

It will be understood by one skilled in the art that the described embodiments herein do not limit the scope of the invention. The specification, including the examples, is intended to be exemplary only, and it will be apparent to those skilled in the art that various modifications and variations can be made in the present invention without departing from the scope or spirit of the invention as defined by the appended claims.

Furthermore, while certain details in the present disclosure are provided to convey a thorough understanding of the invention as defined by the appended claims, it will be apparent to those skilled in the art that certain embodiments may be practiced without these details. Moreover, in certain instances, well-known methods, procedures, or other specific details have not been described to avoid unnecessarily obscuring aspects of the invention defined by the appended claims.

What is claimed is:

1. A bispecific antibody represented by structural formula (II): [symbol]
wherein:
LCA is a light chain antibody portion:
HCA is a heavy chain antibody portion;
LCB is a light chain antibody portion;
HCB is a heavy chain antibody portion;
L¹, L² and L³ include 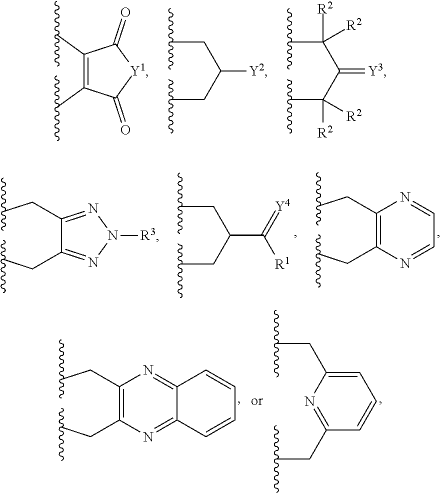
wherein:
Y¹ is O (oxygen), NR⁴, —NH—NH—, or —CH═CH—;
Y² is OH or C₁₋₆ alkoxy;
Y³ is O (oxygen), N—OR⁴, or —CF₂—;
Y⁴ is O (oxygen), N—OR⁴, or —CF₂—;
R¹ is H (hydrogen), C₁₋₆ alkyl, aryl, or heteroaryl;
R² is H (hydrogen) or C₁₋₆ alkyl;
R³ is H (hydrogen) or C₁₋₆ alkyl; and
R⁴ is H (hydrogen) or C₁₋₆ alkyl,
wherein LCA and LCB are not derived from the same antibody.

2. The bispecific antibody of claim 1, wherein HCA includes a heavy chain portion from IgA, IgD, IgE, IgG, or IgM, or up to 10 amino acid replacement sequence thereof or chimera thereof.

3. The bispecific antibody of claim 1 or 2, wherein HCB includes a heavy chain portion from IgA, IgD, IgE, IgG, or IgM, or up to 10 amino acid replacement sequence thereof or chimera thereof.

4. The bispecific antibody of any one of claims 1 to 3, wherein LCA includes a light chain portion from IgA, IgD, IgE, IgG, or IgM, or up to 10 amino acid replacement sequence thereof or chimera thereof.

5. The bispecific antibody of any one of claims 1 to 3, wherein LCB includes a light chain amino portion from IgA, IgD, IgE, IgG, or IgM, or up to 10 amino acid replacement sequence thereof or chimera thereof.

6. The bispecific antibody of any one of claims 1 to 5, wherein LCA, LCB, HCA and HCB each comprise at least one modified L-cysteine-amino acid residue having a carbon-sulfur-carbon bond.

7. The bispecific antibody of any one of claims 1 to 5, wherein at least one sulfur of —S-L¹-S—, —S-L²-S—, or —S-L³-S— is from an L-Cysteine of a peptide before conjugation.

8. The bispecific antibody of any one of claims 1 to 5, wherein —S-L¹-S—, —S-L²-S—, or —S-L³-S— comprise at least one thioether.

9. The bispecific antibody of any one of claims 1 to 5, wherein L¹, L² or L³ includes a 2-carbon bridge, 3-carbon bridge, or 4-carbon bridge.

10. The bispecific antibody of any one of claim 8 or 9, wherein —S-L¹-S—, —S-L²-S—, or —S-L³-S— includes a fragment selected from the group consisting of:

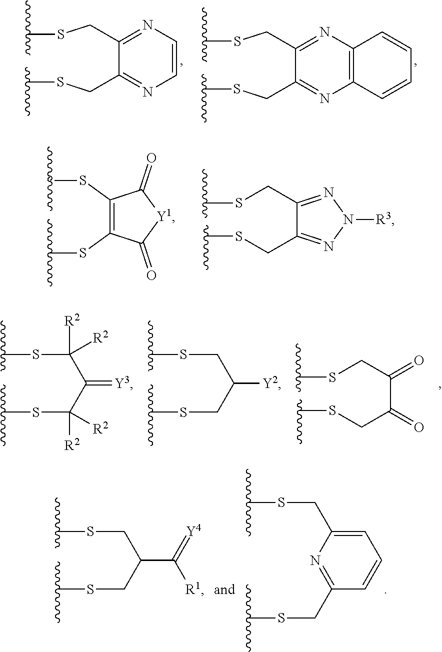

11. The bispecific antibody of any one of claims 1 to 10, wherein HCA and HCB each include the amino acid sequence SPPC, CPPS, APPC or CPPA in the hinge region wherein the cysteine sulfur of the SPPC, CPPS, APPC or CPPA sequence of HCA and HCB is covalently bonded to L³, wherein LCA and LCB are not derived from the same antibody.

12. The bispecific antibody of any one of claims 1 to 11, wherein at least one amino acid residue in the hinge region of HCA or HCB is replaced with at least one cysteine residue.

13. The bispecific antibody of any one of claims 1 to 11, wherein at least one amino acid residue in the hinge region of HCA and HCB is replaced with at least one cysteine residue.

14. The bispecific antibody of claim 12 or 13, wherein at least two amino acid residues in the hinge region of HCA and HCB are each replaced with a cysteine residue.

15. The bispecific antibody of any one of claims 12 to 14, wherein at the hinge region of HCA and/or HCB comprises amino acids 210-250 (EU numbering system).

16. The bispecific antibody of any one of claims 12 to 14, wherein at the hinge region of HCA and/or HCB consists of amino acids 210-250 (EU numbering system).

17. A method of making the bispecific antibody of any one of claims 1 to 16 comprising:
treating [symbol]
with X-L-X for a period of time to provide the bispecific antibody,
wherein X is halo or —OS(O)₂—R⁶;
L is 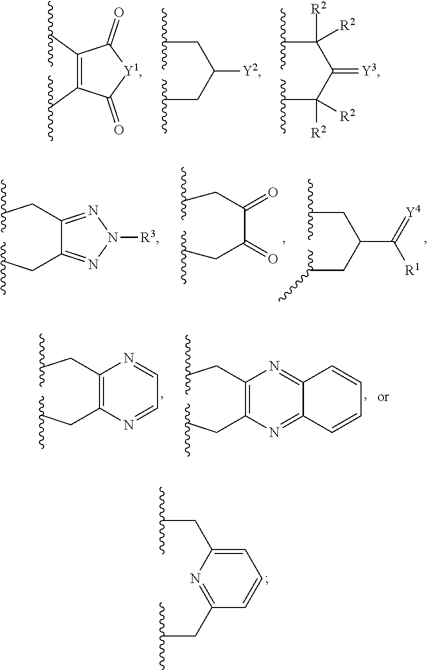
Y¹ is O (oxygen), NR⁴, —NH—NH—, or —CH═CH—;
Y² is OH or C₁₋₆ alkoxy;
Y³ is O (oxygen), N—OR⁴, or —CF₂—;
Y⁴ is O (oxygen), N—OR⁴, or —CF₂—;
R¹ is H (hydrogen), C₁₋₆ alkyl, aryl, or heteroaryl;
R² is H (hydrogen) or C₁₋₆ alkyl;
R³ is H (hydrogen) or C₁₋₆ alkyl;
R⁴ is H (hydrogen) or C₁₋₆ alkyl; and
R⁶ is a synthetic linker optionally substituted C₁₋₆ alkyl, optionally substituted aryl or optionally substituted heteroaryl.

18. The method claim 17, wherein HCA includes a heavy chain portion from IgA, IgD, IgE, IgG, or IgM, or up to 10 amino acid replacement sequence thereof or chimera thereof.

19. The method of claim 17 or 18, wherein HCB includes a heavy chain portion from IgA, IgD, IgE, IgG, or IgM, or up to 10 amino acid replacement sequence thereof or chimera thereof.

20. The method of any one of claims 17 to 19, wherein LCA includes a light chain portion from IgA, IgD, IgE, IgG, or IgM, or up to 10 amino acid replacement sequence thereof or chimera thereof.

21. The method of any one of claims 17 to 20, wherein LCB includes a light chain portion from IgA, IgD, IgE, IgG, or IgM, or up to 10 amino acid replacement sequence thereof or chimera thereof.

22. The method of any one of claims 17 to 21, wherein LCA, LCB, HCA and HCB each comprise at least one modified L-cysteine-amino acid residue having a carbon-sulfur-carbon bond.

23. The method of any one of claims 17 to 22, wherein at least one sulfur of —S-L³-S— is from an L-Cysteine of a peptide before covalently bonding to the linker.

24. The method of any one of claims 17 to 22, wherein —S-L¹-S—, —S-L²-S—, or —S-L³-S— comprise at least one thioether.

25. The bispecific antibody or method of any one of claims 1 to 24, wherein HCA and/or HCB include the amino acid sequence:
EVQLVESGGGLVQPGRSLRLSCAASGFTFDDYAMHWVRQAPGKGLEWVSA
ITWNSGHIDYADSVEGRFTISRDNAKNSLYLQMNSLRAEDTAVYYCAKVS
YLSTASSLDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTSPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFLLYSKLTVDKSRWQQGNVFSCSVMHEALHNHYTQKSLSLS
PGK.

26. The bispecific antibody or method of any one of claims 1 to 24, wherein HCA and/or HCB include the amino acid sequence:
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTSPPCPAPELLGGPSVFLFP
PKPKDTLMISRTPEVTCVVVDVSHEDPEVKFNWYDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFLLYSKLTVDKSRWQQGNVFSCSVMHEALHNHYTQKSLSLSP
GK.

27. The bispecific antibody or method of any one of claims 1 to 24, wherein HCA and/or HCB include the amino acid sequence:
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTSPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSRLTVDKSRWQQGNVFSCSVMHEALHNHYTQKSLSLSPGK.

28. The bispecific antibody or method of any one of claims 1 to 24, wherein HCA and/or HCB include the amino acid sequence:
EVQLVESGGGLVQPGGSLRLSCAASGFTFSNYWMNWVRQAPGKGLEWVAA
INQDGSEKYYVGSVKGRFTISRDNAKNSLYLQMNSLRVEDTAVYYCVRDY
YDILTDYYIHYWYFDLWGRGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKRVEPKSCDKTHTSPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSRLTVDKSRWQQGNVFSCSVMHEALHNHYTQKS
LSLSPGK.

29. The bispecific antibody or method of any one of claims 1 to 24, wherein HCA and/or HCB include the amino acid sequence:
EVQLLESGGGLVQPGGSLRLSCAASGFTFSSYAMSWVRQAPGKGLEWVSY
ISDDGSLKYYADSVKGRFTISRDNSKNTLYLQMNSLRAEDTAVYYCAKHP
YWYGGQLDLWGQGTLVTVSSASTKGPSVFPLAPSSKSTSGGTAALGCLVK
DYFPEPVTVSWNSGALTSGVKTFPAVLQSSGLYSLSSVVTVPSSSLGTQT
YICNVNHKPSNTKVDKRVEPKSCDKTHTSPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEVKCKVSNKALPAPIEKTISKAKGQPREPQ
VYTLPPSREEMTKNQVSLTCLVKGFYPSDIAVEWESNGQPENNYKTTPPV
LDSDGSFFLYSRLTVDKSRWQQGNVFSCSVMHEALHNHYTQKSLSLSPG
K.

30. The bispecific antibody or method of any one of claims 1 to 24, wherein HCA and/or HCB include the amino acid sequence:
QVQLVESGAEVKKPGASVKVSCKASGYTFTGYYMHWVRQAPGQGLEWMG
WINPNSGGTNYAQKFQGRVTMTRDTSISTAYMELSRLRSDDTAVYYCARS
PNPYYYDSSGYYYPGAFDIWGQGTMVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKRVEPKSCDKTHTSPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQP
ENNYKTTPPVLDSDGSFFLYSRLTVDKSRWQQGNVFSCSVMHEALHNHYT
QKSLSLSPGK.

31. The bispecific antibody or method of any one of claims 1 to 24, wherein HCA and/or HCB include the amino acid sequence:
QVQLQESGPGLVKPSETLSLTCTVSGGSISIYYWSWIRQPPGKGLEWIGY
VYYSGSTNYNPSLKSRVTISVDTSKNQFSLKLNSVTAADTAVYYCARGGY
DFWSGYFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTSPPCPAPELLGGPSVFLFPPKP
KDTLMISRTPEVTCVVVDVSHEDPEVKFNWYVDGYEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSRLTVDKSRWQQGNVFSCSVMHEALHNHYTQKSLSLSPG
K.

32. The bispecific antibody or method of any one of claims 1 to 24, wherein HCA and/or HCB include the amino acid sequence:
QVQLVQSGAEVKKPGASVKVSCKASGYTFISYTMHWVRQAPGQGLEWMGY
INPRSGYTHYNQKLKDKATLTADKSASTAYMELSSLRSEDTAVYYCARSA
YYDYDGFAYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTSPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFLLYSKLTVDKSRWQQGNVFSCSVMHEALHNHYTQKSLSLSPG
K.

33. The bispecific antibody or method of any one of claims 1 to 24, wherein HCA and/or HCB include the amino acid sequence:
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTSPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSRLTVDKSRWQQGNYFSCSVMHEALHNHYTQKSLSLSPG
K.

34. The bispecific antibody or method of any one of claims 1 to 24, wherein HCA and/or HCB include the amino acid sequence:
EVQLVESGGGLVQPGGSLRLSCAASGFDLGFYFYACWVRQAPGKGLEWVS
CIYTAGSGSTYYASWAKGRFTISRDNSKNTLYLQMNSLRAEDTAVYYCAR
STANTRSTYYLNLWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCDKTHTSPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSRLTVDKSRWQQGNVFSCSVMHEALHNHYTQKSLSL
SPGK.

35. The bispecific antibody or method of any one of claims 1 to 24, wherein HCA and/or HCB include the amino acid sequence:
EVQLVESGGGLVKPGGSLRLSCAASGFTFSGYGMSWVRQAPGKGLEWVSS
ITSGGTYTYYADSVKGRFTISRDNAKNSLYLQMNSLRAEDTAVYYCARSL
AGNAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTSPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTTSKAKGQPREPQVY
TLPPSREEMTKNQVSLTCLVKGFYPSDIAVEWESNGQPENNYKTTPPVLD
SDGSFLLYSKLTVDKSRWQQGNVFSCSVMHEALHNHYTQKSLSLSPGK.

36. The bispecific antibody or method of any one of claims 1 to 24, wherein HCA and/or HCB include the amino acid sequence:
QVQLVQSGGGLVQPGGSLRLSCAASGFTFSSYEMNWVRQAPGKGLEWVSY
ISSSGSTIYYADSVKGRFTISRDNAKNSLYLQMNSLRAEDTAVYYCAREM
QFGWELLGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTSPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSRLTVDKSRWQQGNVFSCSVMHEALHNHYTQKSLSLS
PGK.

37. The bispecific antibody or method of any one of claims 1 to 24, wherein HCA and/or HCB include the amino acid sequence:
EVQLVESGGGLVQPGGSLRLSCAASGFTFNNYAMSWVRQAPGKGLEWVST
ISSDGTYTYYADSVKGRFTISRDNSKNTLYLQMNSLRAEDTAVYYCARHP
PSYYYAFDYWGQGTTLTVSSASTKGPSVFPLAPSSKSTSGGTAALGCLVK
DYFPEPVTVSWNSGALTSGVHTFPAVLQSSGLVSLSSVVTVPSSSLGTQT
YICNVNHKPSNTKVDKRVEPKSCDKTHTSPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSRLTVDKSRWQQGNVFSCSVMHEALHNHYTQKSLSLSPG
K.

38. The bispecific antibody or method of any one of claims 1 to 24, wherein HCA and/or HCB include the amino acid sequence:
EVQLVESGGGLVQPGGSLRLSCAASGFTFNNAMSWVRQAPGKGLEWVSTI
SSDGTYTYYADSVKGRFTISRDNSKNTLYLQMNSLRAEDTAVYYCARHPP
SYYYAFDYWGQGTTLTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTSPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSRLTVDKSRWQQGNVFSCSVMHEALHNHYTQKSLSLSPGK.

39. The bispecific antibody or method of any one of claims 1 to 24, wherein HCA and/or HCB include the amino acid sequence:
EVQLVESGGGLVQPGGSLRLSCAASGFTFNKNAMNWVRQAPGKGLEWVAR
IRNKTNNYATYYAASVKGRFTISRDDSKSSLYLQMNNLKTEDTAMYYCVA
GNSFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTSPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSRLTVDKSRWQQGNVFSCSVMHEALHNHYTQKSLSLSPGK.

40. The bispecific antibody or method of any one of claims 1 to 24, wherein HCA and/or HCB include the amino acid sequence:
QVQLQESGPGLVKPSETLSLTCVVSGGSISSSNWWSWVRQPPGKGLEWIG
EIYHSGSPDYNPSLKSRVTISVDKSRNQFSLKLSSVTAADTAVYYCAKVS
TGGFFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTSPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSRLTVDKSRWQQGNVFSCSVMHEALHNHYTQKSLSLSPGK.

41. The bispecific antibody or method of any one of claims 1 to 24, wherein HCA and/or HCB include the amino acid sequence:
EVQLVESGGGLVQPGGSLRLSCAASGFTFNKNAMNWVRQAPGKGLEWVAR
IRNKTNNYATYYAASVKGRFTISRDDSKSSLYLQMNNLKTEDTAMYYCVA
GNSFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTSPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSRLTVDKSRWQQGNVFSCSVMHEALHNHYTQKSLSLSPGK.

42. The bispecific antibody or method of any one of claims 1 to 24, wherein LCA and/or LCB include the amino acid sequence:
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPCTFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

43. The bispecific antibody or method of any one of claims 1 to 24, wherein LCA and/or LCB include the amino acid sequence:
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNFYPREAKVQWKVD
NALQSGNSQESVTEQDSKDSTYSLSSTLTLSKADYEKHKVYACEVTHQGL
SSPVTKSFNRGEC.

44. The bispecific antibody or method of any one of claims 1 to 24, wherein LCA and/or LCB include the amino acid sequence:
DIVMTQTPLSLSVTPGQPASISCRSSRSLVHSRGNTYLHWYLQKPGQSPQ
LLIYKVSNRFIGVPDRFSGSGSGTDFTLKISRVEAEDVGVYYCSQSTHLP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

45. The bispecific antibody or method of any one of claims 1 to 24, wherein LCA and/or LCB include the amino acid sequence:
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPCTFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

46. The bispecific antibody or method of any one of claims 1 to 24, wherein LCA and/or LCB include the amino acid sequence:
SYELTQPPSVSVSPGQTASITCSGDSLGSYFVHWYQQKPGQSPVLVIYDD
SNRPSGIPERFSGSNSGNTATLTTSGTQAMDEADYYCSAFTHNSDVFGGG
TKLTVLGQPKAAPSVTLFPPSSEELQANKATLVCLISDFYPGAVTVAWKA
DSSPVKAGVETTTPSKQSNNKYAASSYLSLTPEQWKSHRSYSCQVTHEGS
TVEKTVAPTECS.

47. The bispecific antibody or method of any one of claims 1 to 24, wherein LCA and/or LCB include the amino acid sequence:
QPGLTQPPSVSVAPGQTARITCGGNNIGSKSVHWYQQKPGQAPVLVVYDD
SDRPSGIPERFSGSNSGNTATLTISRVEAGDEADYYCQVWDSSSDHYVFG
TGTKVTVLGQPKAAPSVTLFPPSSEELQANKATLVCLISDFYPGAVTVAW
KADSSPVKAGVETTTPSKQSNNKYAASSYLSLTPEQWKSHRSYSCQVTHE
GSTVEKTVAPTECS.

48. The bispecific antibody or method of any one of claims 1 to 24, wherein LCA and/or LCB include the amino acid sequence:
EIVMTQSPATLSVSPGERATLSCRASQSVDSNLAWYRQKPGQAPRLLIYG
ASTRATGIPARFSGSGSGTEFTLTISSLQSEDFAVYYCQQYINWPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

49. The bispecific antibody or method of any one of claims 1 to 24, wherein LCA and/or LCB include the amino acid sequence:
DIQMTQSPSSLSASVGDRVTITCSASSSVSYMNWYQQKPGKAPKRLIYDT
SKLASGVPSRFSGSGSGTDFTLTISSLQPEDFATYYCQQWSSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

50. The bispecific antibody or method of any one of claims 1 to 24, wherein LCA and/or LCB include the amino acid sequence:
QIVLSQSPAILSASPGEKVTMTCRASSSVSYIHWFQQKPGSSPKPWIYAT
SNLASGVPVRFSGSGSGTSYSLTISRVEAEDAATYYCQQWTSPPTFGGGT
KLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC.

51. The bispecific antibody or method of any one of claims 1 to 24, wherein LCA and/or LCB include the amino acid sequence:
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

52. The bispecific antibody or method of any one of claims 1 to 24, wherein LCA and/or LCB include the amino acid sequence:
DIQMTQSPSSLSASVGDRVTITCQASQRISSYLSWYQQKPGKVPKLLIYG
ASTLASGVPSRFSGSGSGTDFTLTISSLQPEDVATYYCQSYAYFDSNNWH
AFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

53. The bispecific antibody or method of any one of claims 1 to 24, wherein LCA and/or LCB include the amino acid sequence:
EIVLTQSPDFQSVTPKEKVTITCRASQTISDYLHWYQQKPDQSPKLLIKF
ASQSFSGVPSRFSGSGSGTDFTLTINSLEAEDAATYYCQNGHGFPRTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

54. The bispecific antibody or method of any one of claims 1 to 24, wherein LCA and/or LCB include the amino acid sequence:
EIVLTQSPGTLSLSPGERATLSCRASESVDSYGNSFIHWYQQKPGQAPRL
LIYLASNLESGIPDRFSGSGSGTDFTLTISRLEPEDFAVYYCQQNNEDLW
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

55. The bispecific antibody or method of any one of claims 1 to 24, wherein LCA and/or LCB include the amino acid sequence:
DIVMTQSPSSLAVSLGERVTMTCKSSQSLLYSSNQKNYLAVVYQQKPGQS
PKLLIYWASSRESGVPDRFSGSGSGTDFTLTISSVQAEDVAVYYCQQYYN
YPLTFGQGTRLELKRTVAAPSVFIFPPSDEQLKSGTASVVCLLNNFYPRE
AKVQWKVDNALQSGNSQESVTEQDSKDSTYSLSSTLTLSKADYEKHKVYA
CEVTHQGLSSPVTKSFNRGEC.

56. The bispecific antibody of any one of the preceding claims, wherein bispecific antibody targets EGFR/Her2, Her2/Her3, EGFR/cMet, CTLA-4/PD-1, PD-1/TIM-3, PD-1/LAG-3, PD-1/KIR, PD-1/NKD2A, PD-L1/CD47, CD3-CD19, CD3-CD20, CD3-CD33, CD3-CD123, CD3-CD38, NKG2D-Trop2, NKG2D-5T4, NKG2D-GPC3, NKG2D-Mesothelin, NKG2D-WT1, NKG2D/NY-ESO, NKp46-Trop2, NKp46-5T4, NKp46-GPC3, NKp46-Mesothelin, NKp46-WT1, NKp46-ESO, CD47-CD22, CD47-CD33, CD47-CD123, CD3-CD38, CD47-Trop2, CD47-5T4, CD47-GPC3, CD47-Mesothelin, CD47-WT1, or CD47-ESO.

57. The bispecific antibody of any one of claims 42-56, wherein LCA and/or LCB include an amino acid sequence that is at least 90° % identical to that of the recited amino acid sequence.

58. The bispecific antibody of any one of claims 25-41, wherein HCA and/or HCB include an amino acid sequence that is at least 90% identical to that of the recited amino acid sequence.

59. A method of treating cancer, comprising administering a bispecific antibody according to any one of claims 1-58 to a subject in need thereof, wherein the cancer is selected from the group consisting of a carcinoma, a sarcoma, a lymphoma, and a blastoma.

60. A method of treating cancer, comprising administering a bispecific antibody according to any one of claims 1-58 to a subject in need thereof, wherein the cancer is selected from the group consisting of uterine sarcoma cancer, bladder cancer, breast cancer, colon cancer, rectal cancer, endometrial cancer, kidney cancer, lung cancer, melanoma, non-Hodgkin lymphoma, glioblastoma, pancreatic cancer, prostate cancer, ovarian cancer, and thyroid cancer.

61. A method of treating a disease selected from the group consisting of uterine sarcoma cancer, bladder cancer, breast cancer, colon cancer, rectal cancer, endometrial cancer, kidney cancer, lung cancer, melanoma, non-Hodgkin lymphoma, leukemia, pancreatic cancer, prostate cancer, ovarian cancer, and thyroid cancer, comprising administering a bispecific antibody according to any one of claims 1-58 to a subject in need thereof.

62. A method of delivering a bispecific antibody according to any one of claims 1-56 to an in vivo mammalian cell, the method comprising administering a compound of any one of claims 1-58 to a mammal comprising the in vivo mammalian cell.

63. The method of claim 62, wherein the compound is administered parenterally.

64. The method of claim 63, wherein the compound is administered intravenously.

65. The method of claim 62, wherein the compound is administered orally.


SEQUENCE LISTING (30 sequences)

SEQ ID NO: 1 - protein, 451 aa, Artificial Sequence
  Antibody
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 2 - protein, 215 aa, Artificial Sequence
  Antibody
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, Artificial Sequence
  Antibody
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 4 - protein, 214 aa, Artificial Sequence
  Antibody
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Artificial Sequence
  Antibody
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 6 - protein, 219 aa, Artificial Sequence
  Antibody
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 457 aa, Artificial Sequence
  Antibody
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 8 - protein, 215 aa, Artificial Sequence; identical to SEQ ID NO: 2
  Antibody

SEQ ID NO: 9 - protein, 450 aa, Artificial Sequence
  Antibody
EVQLLESGGGLVQPGGSLRLSCAASGFTFSSYAMSWVRQAPGKGLEWVSYISDDGSLKYYADSVKGRFTISRDNSKNTLYLQMNSLRAEDTAVYYCAKHPYWYGGQLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 10 - protein, 212 aa, Artificial Sequence
  Antibody
SYELTQPPSVSVSPGQTASITCSGDSLGSYFVHWYQQKPGQSPVLVIYDDSNRPSGIPERFSGSNSGNTATLTISGTQAMDEADYYCSAFTHNSDVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 459 aa, Artificial Sequence
  Antibody
QVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 12 - protein, 214 aa, Artificial Sequence
  Antibody
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 450 aa, Artificial Sequence
  Antibody
QVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 14 - protein, 215 aa, Artificial Sequence
  Antibody
EIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 450 aa, Artificial Sequence
  Antibody
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 16 - protein, 213 aa, Artificial Sequence
  Antibody
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 451 aa, Artificial Sequence
  Antibody
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 18 - protein, 213 aa, Artificial Sequence
  Antibody
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 448 aa, Artificial Sequence
  Antibody
QVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Artificial Sequence
  Antibody
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 454 aa, Artificial Sequence
  Antibody
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 22 - protein, 218 aa, Artificial Sequence
  Antibody
DIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 448 aa, Artificial Sequence
  Antibody
EVQLVESGGGLVKPGGSLRLSCAASGFTFSGYGMSWVRQAPGKGLEWVSSITSGGTYTYYADSVKGRFTISRDNAKNSLYLQMNSLRAEDTAVYYCARSLAGNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 24 - protein, 214 aa, Artificial Sequence
  Antibody
EIVLTQSPDFQSVTPKEKVTITCRASQTISDYLHWYQQKPDQSPKLLIKFASQSFSGVPSRFSGSGSGTDFTLTINSLEAEDAATYYCQNGHGFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 453 aa, Artificial Sequence
  Antibody
QVQLVQSGGGLVQPGGSLRLSCAASGFTFSSYEMNWVRQAPGKGLEWVSYISSSGSTIYYADSVKGRFTISRDNAKNSLYLQMNSLRAEDTAVYYCAREMQFGWELLGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 26 - protein, 212 aa, Artificial Sequence
  Antibody
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, Artificial Sequence
  Antibody
EVQLVESGGGLVQPGGSLRLSCAASGFTFNNYAMSWVRQAPGKGLEWVSTISSDGTYTYYADSVKGRFTISRDNSKNTLYLQMNSLRAEDTAVYYCARHPPSYYYAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 28 - protein, 218 aa, Artificial Sequence
  Antibody
EIVLTQSPGTLSLSPGERATLSCRASESVDSYGNSFIHWYQQKPGQAPRLLIYLASNLESGIPDRFSGSGSGTDFTLTISRLEPEDFAVYYCQQNNEDLWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 447 aa, Artificial Sequence
  Antibody
EVQLVESGGGLVQPGGSLRLSCAASGFTFNKNAMNWVRQAPGKGLEWVARIRNKTNNYATYYAASVKGRFTISRDDSKSSLYLQMNNLKTEDTAMYYCVAGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 30 - protein, 220 aa, Artificial Sequence
  Antibody
DIVMTQSPSSLAVSLGERVTMTCKSSQSLLYSSNQKNYLAWYQQKPGQSPKLLIYWASSRESGVPDRFSGSGSGTDFTLTISSVQAEDVAVYYCQQYYNYPLTFGQGTRLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC